(19) United States
(12) Patent Application Publication
(10) Pub. No.: US 2025/0319771 A1
(43) Pub. Date: Oct. 16, 2025

(54) ACTION RECOMMENDATION DEVICE, ACTION RECOMMENDATION METHOD, AND RECORDING MEDIUM

(71) Applicant: NEC Corporation, Tokyo (JP)

(72) Inventors: Mana HASHIMOTO, Tokyo (JP); Yuki KOSAKA, Tokyo (JP); Shuhei NOYORI, Tokyo (JP); Isao YAMADA, Tokyo (JP); Takayuki SHIBUYA, Tokyo (JP)

(73) Assignee: NEC Corporation, Tokyo (JP)

(21) Appl. No.: 19/070,625

(22) Filed: Mar. 5, 2025

(30) Foreign Application Priority Data
Apr. 10, 2024 (JP) 2024-063206

(51) Int. Cl. B60K 35/28; B60W 40/08; G16H 20/00

(52) U.S. Cl. CPC B60K 35/28; B60W 40/08; G16H 20/00; B60W 2040/0872; B60W 2540/221

(57) ABSTRACT

Action recommendation device includes at least one memory configured to store instructions; and at least one processor configured to execute the instructions to: acquire information about a life of a disaster victim living an evacuation life in a vehicle, the information being obtained based on a detection result by a sensor installed in the vehicle, determine an action for maintaining health of the disaster victim, the action being recommended to the disaster victim, based on information about the life of the disaster victim, and output information about the action for maintaining health. The action recommendation device makes it possible to support decision making on the action for maintaining health by the disaster victim.

This application is based upon and claims the benefit of priority from Japanese Patent Application No. 2024-063206, filed on Apr. 10, 2024, the disclosure of which is incorporated herein in its entirety by reference.

### TECHNICAL FIELD

The present disclosure relates to an action recommendation device, an action recommendation method, and a program.

### BACKGROUND ART

When a disaster occurs, a disaster victim may evacuate by a vehicle. There is a case where the disaster victim lives an evacuation life in the vehicle.

Reference Document 1 (JP 2023-168077 A) describes a disaster evacuation support device that acquires disaster situation information and disaster victim situation information and generates an evacuation plan.

### SUMMARY

An object of the present disclosure is to provide an action recommendation device or the like that can promote the health of the disaster victim who lives an evacuation life in a vehicle at the time of disaster.

An action recommendation device according to an aspect of the present disclosure includes at least one memory configured to store instructions; and at least one processor configured to execute the instructions to: acquire information about a life of a disaster victim living an evacuation life in a vehicle, the information being obtained based on a detection result by a sensor installed in the vehicle, determine an action for maintaining health of the disaster victim, the action being recommended to the disaster victim, based on information about the life of the disaster victim, and output information about the action for maintaining health.

An action recommendation method according to an aspect of the present disclosure includes acquiring information about a life of a disaster victim living an evacuation life in a vehicle, the information being obtained based on a detection result by a sensor installed in the vehicle, determining an action for maintaining health of the disaster victim, the action being recommended to the disaster victim, based on information about the life of the disaster victim, and outputting information about the action for maintaining health.

A program according to an aspect of the present disclosure causes a computer to execute the steps of acquiring information about a life of a disaster victim living an evacuation life in a vehicle, the information being obtained based on a detection result by a sensor installed in the vehicle, determining an action for maintaining health of the disaster victim, the action being recommended to the disaster victim, based on information about the life of the disaster victim, and outputting information about the action for maintaining health.

The program may be stored in a non-transitory computer-readable recording medium.

### BRIEF DESCRIPTION OF THE DRAWINGS

Exemplary features and advantages of the present disclosure will become apparent from the following detailed description when taken with the accompanying drawings in which:

FIG. 1 is a diagram illustrating an example of a configuration of an action recommendation system including an action recommendation device;

FIG. 2 is a block diagram illustrating an example of a configuration of the action recommendation device;

FIG. 3 is a block diagram illustrating another example of the configuration of the action recommendation device;

FIG. 4 is a flowchart illustrating an operation of the action recommendation device;

FIG. 5 is a block diagram illustrating an example of a configuration of the action recommendation device;

FIG. 6 is a flowchart illustrating an operation of the action recommendation device; and

FIG. 7 is a diagram illustrating an example of a hardware configuration of the action recommendation device.

### EXAMPLE EMBODIMENT

Example embodiments of the present disclosure will be described in detail with reference to the drawings.

### First Example Embodiment

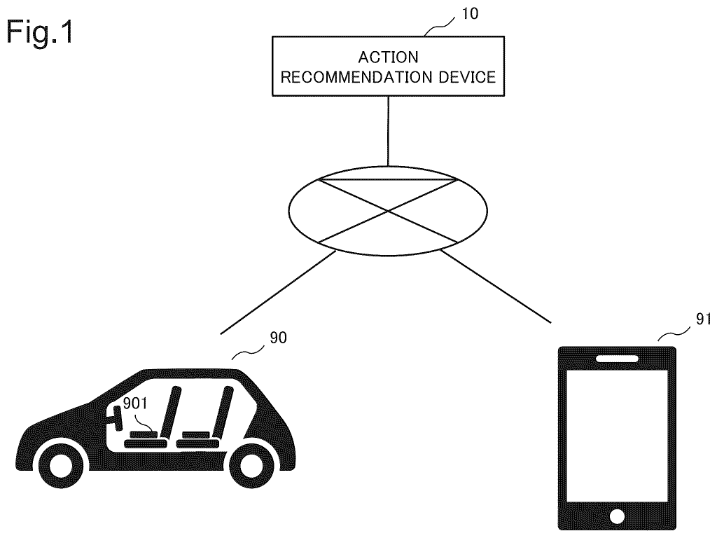

A configuration of an action recommendation system including an action recommendation device 10 will be described with reference to FIG. 1. FIG. 1 is a diagram illustrating an example of a configuration of an action recommendation system including the action recommendation device 10. Referring to FIG. 1, the action recommendation device 10 is connected to a sensor 901 installed in a vehicle 90 via a wired or wireless network.

The vehicle 90 is a vehicle used by a disaster victim. An example of the vehicle 90 is a passenger car. Specifically, the vehicle 90 is a vehicle in which a disaster victim lives an evacuation life. The vehicle 90 is, for example, a private car owned by a disaster victim. The vehicle 90 may be a vehicle prepared to be used by a disaster victim after the disaster occurs. Referring to FIG. 1, the action recommendation device 10 is connected to one vehicle 90. The number of vehicles 90 connected to the action recommendation device 10 is not limited to one. The action recommendation device 10 may be connected to a plurality of vehicles 90. When the action recommendation device 10 is connected to a plurality of vehicles 90, the plurality of vehicles 90 may be vehicles 90 owned by different disaster victims. The plurality of vehicles 90 may be a plurality of vehicles 90 owned by the same disaster victim.

In the following description, the period after the occurrence of a disaster may be referred to as a time of disaster. The normal time represents, for example, a period before occurrence of a disaster or a non-occurrence time of a disaster. The disaster victim may use vehicle 90 also at the normal time. Therefore, the term “a disaster victim at the normal time” refers to a state of a person who will a disaster victim due to the occurrence of a disaster and before the occurrence of the disaster. In the following description, the occupant of the vehicle 90 includes a disaster victim.

The sensor 901 installed in the vehicle 90 is a sensor used for the operation of the vehicle 90 at the normal time. The sensor 901 is, for example, a sensor used for safe traveling of the vehicle 90 before a disaster occurs. The sensor 901 may be, for example, a sensor used to ensure the safety of an occupant in the vehicle 90 before a disaster occurs. Specific examples of the sensor 901 are a load sensor and a capacitance sensor. The load sensor is provided, for example, on a frame of a seat of the vehicle 90. The capacitance sensor is provided on a seat cushion of the vehicle 90. In addition, the sensor 901 may be a pressure sensor that measures the internal pressure of the air bladder provided inside the skin, the pressure sensor constituting the seating face of the seat of the vehicle 90. These sensors 901 are used for the purpose of, for example, detecting an occupant of the vehicle 90 at the normal time. The sensor 901 is not limited thereto. The sensor 901 may be a radar sensor for the purpose of occupant detection or the like. The radar sensor is, for example, a millimeter wave radar.

Here, in the example illustrated in FIG. 1, the sensor 901 is a sensor installed on a seat of the vehicle 90. The sensor 901 is represented by a quadrangle in FIG. 1. The sensor installed in the seat of the vehicle 90 may be built in the seat. Examples of the sensor 901 are not limited thereto.

The sensor 901 may be a camera that records a state when the vehicle 90 travels or an accident occurs. The camera may be a vehicle exterior imaging camera that images the outside of the vehicle 90. The camera may be a vehicle interior imaging camera that images the inside of the vehicle 90. The camera is, for example, a drive recorder. The sensor 901 may be, for example, a sensor installed on a steering wheel of the vehicle 90. The sensor installed on the steering wheel may be, for example, a sensor that measures a pulse rate or the like of the driver of the vehicle 90.

In the example illustrated in FIG. 1, the number of sensors 901 is one. The number of sensors 901 installed in the vehicle 90 is not limited to one. A plurality of sensors 901 may be provided. When the plurality of sensors 901 is provided, the plurality of sensors 901 may include the same type of sensors 901. The plurality of sensors 901 may include different types of sensors 901.

The action recommendation device 10 may be connected to an in-vehicle device (not illustrated) mounted on the vehicle 90. The in-vehicle device is connected to the sensor 901 via a wired or wireless network. Therefore, the in-vehicle device can acquire the detection result by the sensor 901. The in-vehicle device and the action recommendation device 10 are connected by a wired or wireless network. The number of in-vehicle devices is not particularly limited. Here, the action recommendation device 10 may be included in the in-vehicle device as part of the function of the in-vehicle device.

Referring to FIG. 1, the action recommendation device 10 may be connected to a communication terminal 91 via a wired or wireless network. The communication terminal 91 is a terminal that can be used by a disaster victim. The communication terminal 91 is a terminal used by a disaster victim at the normal time. The communication terminal 91 used by the disaster victim and the communication terminal 91 used by the disaster victim at the normal time may be the same or different. The communication terminal 91 is, for example, a smartphone, a tablet terminal, a wearable terminal, or a personal computer. The communication terminal 91 is not limited thereto. The number of communication terminals 91 is not limited to one. The action recommendation device 10 may be connected to a plurality of communication terminals 91. The action recommendation device 10 may be connected to communication terminals 91 of a plurality of disaster victims. The action recommendation device 10 may be connected to a plurality of communication terminals 91 used by one disaster victim.

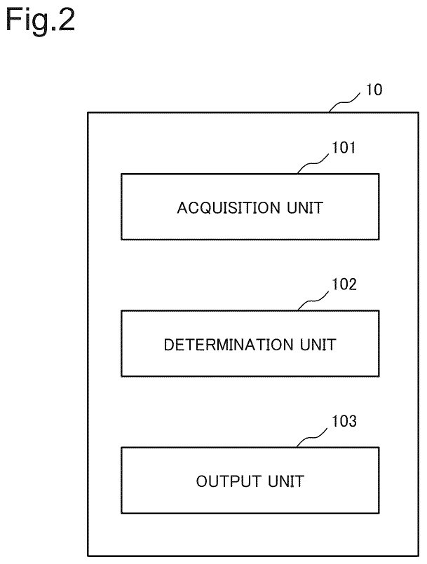

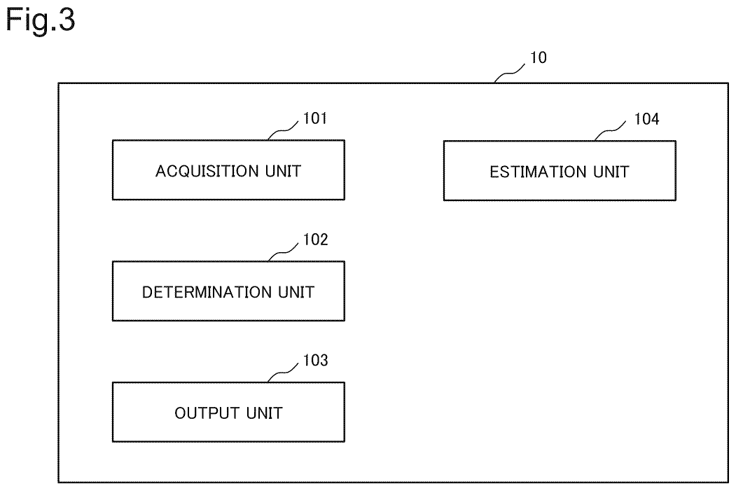

A sensor 911 is installed in the communication terminal 91. The sensor 911 is, for example, an acceleration sensor. The sensor 911 installed in the communication terminal 91 is not limited thereto. A plurality of sensors 911 may be provided. When the plurality of sensors 911 is provided, the plurality of sensors 911 may include the same type of sensors 911. The plurality of sensors 911 may include different types of sensors 911. A configuration of the action recommendation device 10 will be described with reference to FIG. 2. FIG. 2 is a block diagram illustrating an example of a configuration of the action recommendation device 10. The action recommendation device 10 includes an acquisition unit 101, a determination unit 102, and an output unit 103. The action recommendation device 10 may include an estimation unit 104. FIG. 3 is a block diagram illustrating another example of the configuration of the action recommendation device 10. In the example of FIG. 3, the action recommendation device 10 includes an acquisition unit 101, a determination unit 102, an output unit 103, and an estimation unit 104. In the following description, it is assumed that the action recommendation device 10 includes the acquisition unit 101, the determination unit 102, and the output unit 103.

The acquisition unit 101 is an aspect of an acquisition means for acquiring information about the life of a disaster victim living an evacuation life in the vehicle 90, the information being obtained based on a detection result by the sensor 901 installed in the vehicle 90. The acquisition unit 101 analyzes a detection result by the sensor 901 installed in the vehicle 90 at the time of disaster. Then, the acquisition unit 101 acquires information about the life of the disaster victim by analyzing the detection result. Here, the analysis of the detection result by the sensor 901 may be performed by a component different from the acquisition unit 101. For example, the analysis unit may analyze the detection result by the sensor 901. At this time, the acquisition unit 101 can acquire information about the life of the disaster victim by acquiring the analysis result by the analysis unit. The acquisition unit 101 may acquire the detection result by the sensor 901 as information about the life of the disaster victim. The acquisition unit 101 may not analyze the detection result by the sensor 901.

The information about the life of the disaster victim is information about the evacuation life of the disaster victim. That is, the information about the life of the disaster victim is information indicating how the disaster victim spends in the evacuation place. The information about the life of the disaster victim includes an action history in the vehicle 90. The action history in the vehicle 90 is a record of an action of a disaster victim in the vehicle 90. The action history in the vehicle 90 indicates how the disaster victim living an evacuation life in the vehicle 90 spends in the vehicle.

The action history in the vehicle 90 includes, for example, a posture in the vehicle. The posture in the vehicle is, for example, a sitting position or a lying position of an occupant on a seat of the vehicle 90. The action history in the vehicle 90 may include a habit of a posture in the vehicle. The habit of the posture in the vehicle is the tendency of the posture of the occupant when the occupant takes the same posture in the vehicle. One example of the posture habit is that the disaster victim is sitting forward in the sitting position. Another example of the habit of posture is that the disaster victim sits down in such a way as to lean on either the left or the right in the sitting position. Furthermore, another example of the habit of posture is that the disaster victim goes to sleep facing either the left or the right in lying.

The acquisition unit 101 can acquire, for example, a posture or a habit of a posture in the vehicle obtained based on a detection result by a sensor for the purpose of occupant detection. That is, the acquisition unit 101 can acquire information about the posture or the habit of the posture in the vehicle. Here, the sensor for the purpose of occupant detection may be used, for example, for the purpose of preventing the occupant from dozing and driving at the normal time. For example, the load sensor, the capacitance sensor, or the pressure sensor included in the sensor 901 can detect a load distribution or a pressure distribution on the seat face, the backrest, or the frame. Then, the posture or the habit of the posture in the vehicle is estimated by the learned model using the load distribution or the pressure distribution detected by the sensor 901 as an input. The acquisition unit 101 can acquire an estimation result of the posture or the habit of the posture in the vehicle by the learned model.

The acquisition unit 101 may acquire the posture or the habit of the posture in the vehicle from the detection result by the radar sensor. The posture or the habit of the posture in the vehicle is estimated by the learned model using the point cloud data collected by the radar sensor as an input. Then, the acquisition unit 101 can acquire an estimation result of the posture or the habit of the posture in the vehicle by the learned model.

Furthermore, the acquisition unit 101 may acquire the posture or the habit of the posture in the vehicle using the image captured by the vehicle interior imaging camera. Here, the image includes a moving image. The image may be a set of images captured at predetermined time intervals. The predetermined time is a preset time. The joint key points of the body of the occupant are estimated by the learned model using the image captured by the vehicle interior imaging camera as an input. Furthermore, the posture or the habit of the posture in the vehicle is estimated by the learned model using the joint key points of the body of the occupant as inputs. The acquisition unit 101 can acquire an estimation result of the posture or the habit of the posture in the vehicle by the learned model. The learning model for estimating the joint key point and the learned model for estimating the posture or the habit of the posture in the vehicle may be different learned models, or may be the same learned model.

The above-described learned model may be included in the acquisition unit 101. The analysis of the detection result by the sensor 901 using the learned model may be performed by the analysis unit described above. Furthermore, the learned model may be included in an information processing device (not illustrated) different from the action recommendation device 10. At this time, the acquisition unit 101 can acquire an analysis result performed in the information processing apparatus. The action recommendation device 10 is connected to the information processing device via a wired or wireless network.

The action history in the vehicle 90 may include a sitting position retention time in the vehicle. The sitting position retention time in the vehicle is a time during which seating of an occupant is detected. For detection of seating of an occupant, for example, at least one of a load sensor, a capacitance sensor, and a pressure sensor is used. A radar sensor or a vehicle interior imaging camera may be used to detect seating of an occupant. The acquisition unit 101 acquires the time during which the seating of the occupant is detected by these sensors as the sitting position retention time.

The action history in the vehicle 90 may include the amount of physical activity during sitting position retention. The amount of physical activity during the sitting position retention is, for example, a change in the position of the center of gravity of the occupant in the sitting position. The acquisition unit 101 can use, for example, a detection result by at least one of a load sensor, a capacitance sensor, a pressure sensor, a radar sensor, and a vehicle interior imaging camera. The acquisition unit 101 acquires the fluctuation in the position of the center of gravity of the occupant, which is obtained based on the detection result, as the physical activity amount during the sitting position retention.

The action history in the vehicle 90 may include vital signs. An example of a vital sign is heart rate. Another example of the vital sign is respiratory rate. Vital signs are not limited thereto. The acquisition unit 101 can acquire a vital sign obtained based on a detection result by the sensor 901 installed in the vehicle 90. When the sensor 901 is a load sensor, the load sensor detects a ballistocardiogram. The acquisition unit 101 can acquire the heart rate estimated from the detection result by the ballistocardiogram by the load sensor. The acquisition unit 101 can acquire the respiratory rate estimated from the detection result by the ballistocardiogram by the load sensor. The heart rate or the respiratory rate may be estimated by the analysis unit. For example, the analysis unit applies Fourier transform to a signal detected by the load sensor. Then, the analysis unit can estimate the respiratory rate assuming that a peak of a frequency close to the respiratory cycle is a peak of variation associated with respiration.

In addition, the acquisition unit 101 may acquire the vital sign from detection results of a capacitance sensor, a pressure sensor, and a radar sensor. The heart rate or the respiratory rate is estimated based on a periodic variation associated with the heart beat or the respiration in the signal of the sensor 901.

The action history in the vehicle 90 may include a heart rate fluctuation or a pulse fluctuation. The heart rate fluctuation is a fluctuation in the heartbeat interval of the heart. The pulse fluctuation is a fluctuation in pulse interval. The heart rate fluctuation and the pulse fluctuation may be used as indices of autonomic nerve function and stress. The acquisition unit 101 can acquire the heart rate fluctuation or the pulse fluctuation obtained based on the detection result by the sensor 901 installed in the vehicle 90. The signal of the sensor 901 includes a peak of a heart beat or a pulse from fluctuations caused by the heart beat. Therefore, the heart rate fluctuation or the pulse fluctuation is estimated based on the interval between peaks. The acquisition unit 101 can acquire the heart rate fluctuation or the pulse fluctuation estimated based on the interval between the peaks. The analysis unit may estimate the heart rate fluctuation or the pulse fluctuation.

The action history in the vehicle 90 is not limited thereto. The method of acquiring the action history in the vehicle 90 is not limited thereto.

The information about the life of the disaster victim may include, for example, an action history outside the vehicle 90. The action history outside the vehicle 90 is, for example, a record of an action of a disaster victim around the vehicle. An example of the action of the disaster victim around the vehicle is the action in which the disaster victim gets off the vehicle 90 and perform stretching. Acquisition unit 101 may acquire an action history outside vehicle 90 using, for example, an image captured by a vehicle exterior imaging camera as the sensor 901 installed in the vehicle 90. The vehicle exterior imaging camera images the action of the disaster victim within a range that can be imaged by the camera outside the vehicle. Here, as described above, the image includes a moving image. The image may be a set of images captured at predetermined time intervals. The vehicle exterior imaging camera may be used for the purpose of imaging vehicles in front of and behind the vehicle 90 at the normal time.

For example, the acquisition unit 101 may acquire the posture or the habit of the posture of the motion of the disaster victim outside the vehicle. An example of the posture of the motion of the disaster victim outside the vehicle is that the posture when walking is a hunched back. The method of estimating the posture or the habit of the posture of the motion of the disaster victim is similar to the method of estimating the posture or the habit of the posture in the vehicle. The posture or the habit of the posture of the motion may be estimated by the analysis unit.

The information about the life of the disaster victims is not limited thereto. The information about the life of the disaster victim may include the physical condition of the disaster victim. An example of the physical condition is the condition of the joint or the muscle of the disaster victim. The physical condition may be, in particular, the condition of the joint or the muscle of the disaster victim when taking a specific posture or motion. The state of the joint is, for example, a movable range of the joint. The specific posture or motion may be predetermined. The acquisition unit 101 can acquire the physical condition of the disaster victim using, for example, an image captured by a vehicle exterior imaging camera as the sensor 901 installed in the vehicle 90. The vehicle exterior imaging camera captures an image when the disaster victim takes a specific posture or motion. Then, the state of the joint or the muscle of the disaster victim is estimated using the image captured by the vehicle exterior imaging camera. The image includes a moving image. The method of estimating the state of the joint or the muscle is performed using a known technique. For example, the state of the joint or the muscle of the disaster victim is estimated from the spinal column shape of the disaster victim included in the moving image. The state of the joint or the muscle may be estimated by the analysis unit.

The information about the life of the disaster victim may include the mental condition of the disaster victim. The mental condition is, for example, a feeling or a stress of the disaster victim. The feeling or the stress of the disaster victim is estimated based on, for example, vital signs of the disaster victim. The feeling or the stress of the disaster victim is estimated based on, for example, the heart rate fluctuation or the pulse fluctuation of the disaster victim. In addition, the feeling or the stress of the disaster victim may be estimated from the expression of the disaster victim imaged by the vehicle interior imaging camera. A known technique is used to estimate the feeling or the stress of the disaster victim. The analysis unit may estimate the feeling or the stress of the disaster victim.

The information about the life of the disaster victim may include information about the vehicle interior environment. The vehicle interior environment includes information about the temperature or the humidity in the vehicle 90. The acquisition unit 101 can acquire information about the vehicle interior environment using the thermometer or the hygrometer of the vehicle 90 as the sensor 901 installed in the vehicle 90. The thermometer or the hygrometer of the vehicle 90 may be a sensor used to keep a vehicle interior environment comfortable at the normal time.

At the normal time, the acquisition unit 101 may acquire information about the life of the disaster victim obtained based on the detection result by the sensor 901 installed in the vehicle 90. The information about the life of the disaster victim at the normal time is information about the life in the vehicle when the disaster victim uses the vehicle 90 at the normal time. The information about the life of the disaster victim at the normal time includes at least one of an action history in the vehicle 90, an action history outside the vehicle 90, a physical condition, a mental condition, and a vehicle interior environment.

The action history in the vehicle 90 at the normal time is a record of an action in the vehicle 90 when the disaster victim uses the vehicle 90 at the normal time. The action history outside the vehicle 90 at the normal time is a record of the action around the vehicle when the disaster victim uses the vehicle 90 at the normal time. The case where the disaster victim uses the vehicle 90 at the normal time includes a case where the disaster victim is in the vehicle 90 at the normal time. The case where the disaster victim uses the vehicle 90 at the normal time may include, for example, a case where the disaster victim performs maintenance or cleaning of the vehicle 90 at the normal time. The content of each of the physical condition, the mental condition, and the vehicle interior environment is the same as that in the above-described method. A method for acquiring each of the action history in the vehicle 90, the action history outside the vehicle 90, the physical condition, the mental condition, and the vehicle interior environment are similar to the method described above.

The acquisition unit 101 can acquire information about living of a plurality of disaster victims. In the vehicle 90, there is a case where a plurality of disaster victims lives an evacuation life. Even at the normal time, there is a case where a plurality of disaster victims uses the vehicle 90. A seat on which a plurality of occupants is seated may not be constant. Therefore, the acquisition unit 101 can distinguish and acquire information about a plurality of occupants based on the detection result by the sensor 901 installed in the vehicle 90.

A case where the acquisition unit 101 uses, for example, a load sensor or a pressure sensor as a sensor installed in the vehicle 90 will be described. The acquisition unit 101 can distinguish the occupant using the weight of the occupant. The acquisition unit 101 can distinguish the occupant from the difference in the weight of the occupant. The acquisition unit 101 can associate the action history of each occupant even when the seating position of the occupant changes.

In a case where the acquisition unit 101 acquires the physical condition, the occupant can be distinguished from an image obtained by capturing the occupant outside the vehicle. A known technique can be used to distinguish the occupant included in the image. For example, the weight of the occupant is estimated by a machine learning model using an image obtained by capturing an occupant outside the vehicle 90 as an input. Then, the acquisition unit 101 can associate the action history and the physical condition of the occupant.

The detection by the sensor 901 installed in the vehicle 90 may be continuously performed. The detection by the sensor 901 installed in the vehicle 90 may be periodically performed. The timing of detection by the sensor 901 may be different between the time of disaster and the normal time, or may be the same. For example, detection may be continuously performed at the time of disaster, while detection may be performed at predetermined intervals at the normal time. In a case where detection is performed at predetermined intervals during a disaster and during a normal time, the detection intervals may be different between the disaster time and the normal time. At the time of disaster, the fuel of the vehicle 90 may run short. Therefore, the detection interval at the time of disaster may be longer than the detection interval at the normal time.

The timing at which the sensor 901 analyzes the detection result is not particularly limited. For example, the detection result by the sensor 901 may be analyzed in real time. For example, detection results for one day by the sensor 901 may be collectively analyzed.

The acquisition unit 101 may store information obtained from the detection result by the sensor 901 installed in the vehicle 90 in, for example, a database. When the analysis unit analyzes the detection result by the sensor 901 installed in the vehicle 90, the analysis unit may store the analysis result in the database. Then, the determination unit 102 or the estimation unit 104 to be described later can refer to the information stored in the database as necessary.

The acquisition unit 101 may acquire information about the life of the disaster victim obtained from a detection result by a sensor different from the sensor installed in the vehicle 90. At the time of disaster, the acquisition unit 101 can acquire information about the life of the disaster victim obtained based on the detection result by the sensor 911 installed in the communication terminal 91 used by the disaster victim.

The information about the life of the disaster victim includes, for example, an action history outside the vehicle 90. Examples of the action history outside the vehicle 90 include the position information and the number of steps of the disaster victim. The acquisition unit 101 can acquire the action history of the disaster victim by analyzing the detection result by the sensor 911 installed in the communication terminal 91. The analysis of the detection result may be performed by the analysis unit described above. The acquisition unit 101 may acquire, from the communication terminal 91, an action history of the disaster victim, which is a result of analysis by the communication terminal 91. The acquisition unit 101 can acquire an action history of a disaster victim obtained based on a detection result by the sensor 911 installed in the communication terminal 91 at the time of disaster and at the normal time.

The acquisition unit 101 may acquire basic information about the disaster victim. The basic information about the disaster victim includes the attribute information or the medical history of the disaster victim. The attribute information about the disaster victim includes, for example, at least one of the age, sex, family structure, and residential area of the disaster victim. The medical history of the disaster victim includes information about a disease that the disaster victim currently suffers from and a disease that the disaster victim suffered from in the past. The medical history of the disaster victim may include a medication history. The acquisition unit 101 can acquire the basic information about the disaster victim from the communication terminal 91. The disaster victim can input his/her basic information to the communication terminal 91. Then, the acquisition unit 101 can acquire the basic information input by the disaster victim from the communication terminal 91. The acquisition of the basic information by the acquisition unit 101 may be performed at the normal time or at the time of disaster.

The acquisition unit 101 may acquire environmental information outside the vehicle at the time of disaster and at the normal time from the communication terminal 91. The vehicle exterior environment at the time of disaster is an environment outside the vehicle 90 where the disaster victim lives an evacuation life. The vehicle exterior environment at the normal time is an environment outside the vehicle 90 used by the disaster victim at the normal time. The vehicle exterior environment includes at least one of weather, temperature, and humidity. The vehicle exterior environment at the time of disaster includes at least one of disaster occurrence information and a damage situation due to the disaster. The disaster occurrence information is information about a disaster that has occurred around the vehicle. The disaster information includes at least one of the type of disaster that occurred, the date and time of occurrence, and the degree of disaster. The damage situation due to the disaster represents the type and degree of damage caused by the disaster. The damage caused by the disaster includes damage related to transportation infrastructure, damage related to living infrastructure, damage related to houses, and the like. The degree of damage includes a range in which damage has occurred. The acquisition unit 101 can acquire environmental information outside the vehicle from the communication terminal 91. The acquisition unit 101 may acquire the environmental information outside the vehicle from, for example, a database. Here, for example, information about weather, temperature, and humidity may be managed in the database, and disaster occurrence information or information of a damage situation due to a disaster may be stored in the database.

The detection by the sensor 911 installed in the communication terminal 91 may be continuously performed. The detection by the sensor 911 installed in the communication terminal 91 may be periodically performed. The timing of detection by the sensor 911 installed in the communication terminal 91 may be different between the time of disaster and the normal time, or may be the same. For example, detection may be continuously performed at the time of disaster, while detection may be performed at predetermined intervals at the normal time.

Furthermore, the timing at which the detection result by the sensor 911 installed in the communication terminal 91 is analyzed is not particularly limited. For example, the detection result by the sensor 911 installed in the communication terminal 91 may be analyzed in real time. For example, detection results for one day by the sensor 911 installed in the communication terminal 91 may be analyzed collectively.

The acquisition unit 101 may store information obtained from the detection result by the sensor 911 installed in the communication terminal 91 in, for example, a database. When the analysis unit analyzes the detection result by the sensor 911 installed in the communication terminal 91, the analysis unit may store the analysis result in the database. Then, the determination unit 102 or the estimation unit 104 to be described later can refer to the information stored in the database as necessary.

The acquisition unit 101 may acquire information different from the information about the life of the disaster victim, in addition to the information about the life of the disaster victim.

Acquisition unit 101 may further acquire information about a status of use of vehicle 90 at the normal time. The status of use of the vehicle 90 at the normal time includes, for example, an operation time or an operation frequency at normal time. The status of use of the vehicle 90 at the normal time includes a break time or a break acquisition interval acquired during driving. The status of use of the vehicle 90 at the normal time may represent an allowable range of activities of the disaster victim in the vehicle at the normal time. For example, a disaster victim who drives for a long time at the normal time may be less likely to feel stress for the evacuation life in the vehicle 90 than a disaster victim who hardly drives at the normal time. At the normal time, a disaster victim who frequently takes a break while driving may want to frequently go out of the vehicle and move his/her body even when he/she lives an evacuation life in the vehicle 90.

The acquisition unit 101 can acquire the status of use of the vehicle 90 at the normal time using the sensor 901 installed in the vehicle 90. For example, the acquisition unit 101 analyzes a status of use of the vehicle 90 at the normal time based on an image captured by the vehicle interior imaging camera of the vehicle 90. Then, the acquisition unit 101 can acquire an analysis result of the status of use of the vehicle 90. A known technique can be used to analyze the status of use of the vehicle 90. The acquisition unit 101 can analyze the status of use of the vehicle 90 at the normal time based on the operation state of the engine of the vehicle 90. Then, the acquisition unit 101 can acquire an analysis result of the status of use of the vehicle 90. For example, the acquisition unit 101 may set the time during which the engine is operating as the operating time at the normal time. In addition, the acquisition unit 101 may analyze the status of use of the vehicle 90 based on the position information about the vehicle 90. Then, the acquisition unit 101 can acquire an analysis result of the status of use of the vehicle 90. For example, when the vehicle 90 is stopped in a service area of an expressway for a predetermined time, the acquisition unit 101 can analyze the stop time as a break time. A known technique can be used to analyze the status of use of the vehicle 90. The analysis of the status of use of the vehicle 90 may be performed by the analysis unit described above.

The acquisition unit 101 may acquire the status of use of the vehicle 90 at the normal time using the sensor 911 installed in the communication terminal 91. The acquisition unit 101 may acquire the status of use of the vehicle 90 at the normal time from the input of the disaster victim to the communication terminal 91. The acquisition unit 101 may further acquire information about an article that can be used at the time of disaster. Specifically, the acquisition unit 101 can acquire information about an article that can be used by the disaster victim when the disaster victim conducts recommended actions. The article that can be used at the time of disaster is, for example, an article that assists exercise when a disaster victim exercises. The information about the article usable at the time of disaster includes the content of the article and the position of the article.

The acquisition unit 101 acquires information about an article in the vehicle or around the vehicle using the sensor 901 installed in the vehicle 90. The acquisition unit 101 can acquire information about an article that can be used at the time of disaster by analyzing an image captured by a camera installed in the vehicle 90. The camera installed in the vehicle 90 includes at least one of a vehicle exterior imaging camera and a vehicle interior imaging camera. The acquisition unit 101 can acquire information about an article around the vehicle from an analysis result of an image captured by the vehicle exterior imaging camera. The acquisition unit 101 can acquire at least information about an article in the vehicle from an analysis result of an image captured by the vehicle interior imaging camera. A known technique can be used to analyze an image captured by a camera installed in the vehicle 90. The analysis of the image captured by the camera installed in the vehicle 90 may be performed by the analysis unit described above.

The acquisition unit 101 can further acquire information about an article usable at the time of disaster from the communication terminal 91. The acquisition unit 101 may acquire information about an article that can be used at the time of disaster, the information being obtained based on an image captured by a camera installed in the communication terminal 91. The acquisition unit 101 can acquire information about an article usable at the time of disaster by analyzing an image captured by a camera installed in the communication terminal 91. A known technique can be used to analyze an image captured by a camera installed in the communication terminal 91. The analysis of the image captured by the camera installed in the communication terminal 91 may be performed by the analysis unit described above.

The acquisition unit 101 may acquire information about an article that can be used at the time of disaster based on the input of the disaster victim to the communication terminal 91. For example, the disaster victim can input information about an article usable at the time of disaster to the communication terminal 91. Therefore, the acquisition unit 101 acquires information about an article that can be used at the time of disaster input by the disaster victim.

The information about an article that can be used at the time of disaster acquired by the acquisition unit 101 is stored in a database in the form of a list, for example.

Then, the list stored in the database is referred to by the determination unit 102 when the determination unit 102 to be described later determines a motion.

In addition, the acquisition unit 101 can acquire, from the communication terminal 91, community information about a community to which the disaster victim belongs. For example, there is a case where a disaster victim belongs to a local community or the like at the normal time. Therefore, the acquisition unit 101 acquires community information about a community to which the disaster victim belongs. The community information is information about the frequency of participation of the disaster victim in the community and the members belonging to the same community. The acquisition unit 101 may acquire information about an article that can be used at the time of disaster, the information being obtained based on the position information of the communication terminal 91. The acquisition unit 101 may acquire community information by inputting a disaster victim into the communication terminal 91. At this time, the community information may include activity content of the community.

The determination unit 102 is an aspect of a determination means for determining an action for maintaining health of the disaster victim recommended to the disaster victims based on information about life of the disaster victims.

The action for maintaining health of the disaster victim is an action performed by the disaster victim who lives an evacuation life in the vehicle 90 so as not to impair the health. The action for maintaining health of the disaster victim is an action for promoting the health of the disaster victim who lives an evacuation life in the vehicle 90. Specific examples of the action for maintaining health of the disaster victim include stretching of limbs, movement from the inside of the vehicle to the outside of the vehicle, ingestion of food and drink, operation for improving the environment inside the vehicle, and exercise.

Here, a method of determining an action for maintaining health of the disaster victim by the determination unit 102 will be described. The action recommendation device 10 may include an estimation unit 104.

The estimation unit 104 is an aspect of an estimation means for estimating the health condition of the disaster victim based on information about the life of the disaster victim. Then, the determination unit 102 can determine an action according to the health condition estimated by the estimation unit 104. Therefore, after the estimation unit 104 is described, the determination unit 102 will be described.

The estimation unit 104 estimates the health condition of the disaster victim at the time of disaster. The health condition of the disaster victim represents the physical condition of the disaster victim who is living an evacuation life in the vehicle 90.

Specific examples of the health condition include a risk of pain in the musculoskeletal system, a risk of disorder in the musculoskeletal system, disorder in motion of joints and the like, and mental stress. Examples of the risk of pain in the musculoskeletal system include the probability of occurrence of low back pain, the probability of occurrence of shoulder pain, and the probability of occurrence of neck pain. Examples of the health condition are not limited thereto. The health condition may be a risk of exacerbation of a chronic disease or a risk of morbidity of a disease.

The health condition is represented by, for example, a probability. For example, the degree of the risk of disorder of the musculoskeletal system may be represented by a probability. The health condition may be represented by ranks or scores divided into a plurality of stages. For example, mental stress may be represented by a rank divided by the intensity of stress. Furthermore, the health condition may be represented by the presence or absence of risk, disorder, or stress. In addition, the health condition may be represented by a degree of change. For example, the risk of exacerbation of chronic diseases may be expressed by the magnitude of change in a case where the risk of exacerbation is increased. The health condition may be represented by a change rate within a specific period.

The estimation unit 104 may estimate the health condition of the disaster victim at the normal time based on information about the life of the disaster victim.

A method of estimating the health condition of the disaster victim by the estimation unit 104 will be described. The estimation unit 104 estimates the health condition of the disaster victim using the learned model. Here, a learned model used for estimating the health condition of the disaster victim is referred to as a health condition estimation model. The health condition estimation model is a model obtained by learning, by machine learning, the relationship between information about the life of the disaster victim and the health condition of the disaster victim. The estimation unit 104 uses the information about the life of the disaster victim acquired by the acquisition unit 101 as an input to the health condition estimation model. Then, the estimation unit 104 acquires an estimation result of the health condition of the disaster victim, which is an output of the health condition estimation model. The estimation unit 104 may estimate the health condition of the disaster victim using a predetermined algorithm.

When the estimation unit 104 estimates the health condition of the disaster victim at the time of disaster, the input information about the life of the disaster victim includes the information at the time of disaster. When the estimation unit 104 estimates the health condition of the disaster victim at the time of disaster, the input information about the life of the disaster victim may include information at the normal time. The information about the life of the disaster victims at the normal time is used to estimate the health condition of the disaster victim at the time of disaster, so that the estimation unit 104 can estimate the health condition of the disaster victim at the time of disaster based on the health condition at the normal time. When the estimation unit 104 estimates the health condition of the disaster victim at the normal time, the input information about the life of the disaster victim is information at the normal time.

The estimation unit 104 uses, for example, a posture in the vehicle, a habit of the posture, a sitting position retention time in the vehicle, and a physical activity amount during the sitting position retention as inputs. At this time, the estimation unit 104 can estimate the pain risk of the musculoskeletal system using a health condition estimation model or a predetermined algorithm. In addition, the estimation unit 104 uses, for example, a vital sign, a heart rate fluctuation, an emotion, and a stress as inputs. At this time, the estimation unit 104 can estimate the mental stress using a health condition estimation model or a predetermined algorithm. The heart rate fluctuation may be a pulse fluctuation. Furthermore, for example, the estimation unit 104 uses the state of the joint or the muscle as an input when taking a specific posture or motion. At this time, the estimation unit 104 can estimate disorder of the motion of the joint using a health condition estimation model or a predetermined algorithm. Furthermore, the estimation unit 104 can estimate the pain risk of the musculoskeletal system using the vital sign, the heart rate fluctuation, the emotion, and the stress as inputs in addition to the posture in the vehicle, the habit of the posture, the sitting position retention time in the vehicle, and the physical activity amount during the sitting position retention. This is because the feeling of the disaster victim for pain changes due to mental stress and the like in addition to the state of the musculoskeletal system.

For the health condition estimation model or the predetermined algorithm, a known technique can be used. The health condition estimation model may be a model additionally learned for estimation of the health condition.

The health condition estimation model may be a model additionally learned for importance estimation of the importance level used for estimation of the health condition. For example, the importance level of information used for estimation of the health condition is estimated by the additionally learned health condition estimation model. The information used for estimating the health condition is information about the life of the disaster victim at the time of disaster. The information about the life of the disaster victim includes at least one of the action history of the disaster victim in the vehicle 90, the action history of the disaster victim outside the vehicle 90, and the physical condition or mental condition of the disaster victim. The information used for estimating the health condition may include information about the life of the disaster victim at the normal time. The importance level of each of the above information is estimated by the additionally learned health condition estimation model. The importance level is determined by repeating estimation of the health condition by the health condition estimation model.

Here, an example of a method of estimating the importance level will be described. A case where the importance level of the data X is estimated will be described as an example. The health condition estimated by the estimation unit 104 using the data X is compared with the health condition S′ estimated by the estimation unit 104 without using the data X. In a case where S and S′ are close values, the importance level of the data X with respect to the estimation of the health condition is estimated to be small. For example, an absolute error, a square error, or a cross entropy error between S and S′ is used as an index of the importance level.

Another example of a method of estimating the importance level will be described. The importance level of the information can be estimated by using the health condition input by the disaster victim. The estimation unit 104 estimates the health condition of the disaster victim using the information about the life of the disaster victim acquired by the acquisition unit 101. By comparing the estimated health condition with the health condition input by the disaster victim, the importance level of the information can be estimated. Here, the input of the health condition by the disaster victim can be acquired by the acquisition unit 101 via, for example, a device installed in the vehicle 90 or the communication terminal 91.

The information to be acquired by the acquisition unit 101 is determined by the health condition estimation model additionally learned about the importance level estimation of the information. The data acquired by the acquisition unit 101 is determined according to the importance level of the information. For example, the acquisition unit 101 may acquire information with a high importance level and may not acquire information with a low importance level.

The health condition estimation model may be a health condition estimation model that has additionally learned a correlation between information obtained from a detection result by the sensor 901 installed in the vehicle 90 and information obtained from a detection result by the sensor 911 installed in the communication terminal 91 at the normal time. At the normal time, the estimation unit 104 acquires the posture of the occupant, the habit of the posture, the sitting position retention time in the vehicle, and the physical activity amount during the sitting position retention obtained based on the detection result by the sensor 901 installed in the vehicle 90. The acquisition unit 101 acquires the posture of the occupant, the habit of the posture, the sitting position retention time in the vehicle, and the physical activity amount during the sitting position retention obtained based on the detection result by the sensor 911 installed in the communication terminal 91. Then, a correlation coefficient between information obtained based on a detection result by the sensor 901 installed in the vehicle 90 and information obtained based on a detection result by an acceleration sensor or the like installed in the communication terminal 91 is calculated.

Then, at the time of disaster, the information acquired by the acquisition unit 101 is determined by the health condition estimation model additionally learned about the correlation. The acquisition unit 101 acquires, for example, information having a strong correlation from one of a sensor installed in the vehicle 90 and a sensor 911 installed in the communication terminal 91. This is because the information with strong correlation is information from which similar information can be obtained from either the sensor installed in the vehicle 90 or the sensor 911 installed in the communication terminal 91. The acquisition unit 101 may select a sensor that acquires the information in accordance with the remaining battery amount or the power consumption for the information having a strong correlation. The acquisition unit 101 may switch a sensor that acquires information according to a remaining battery amount or power consumption. The plurality of pieces of information having a strong correlation is acquired from either the sensor installed in the vehicle 90 or the sensor 911 installed in the communication terminal 91, whereby power consumption and calculation cost can be reduced.

The determination unit 102 will be described again. The determination unit 102 determines an action for maintaining health of the disaster victim based on the health condition of the disaster victim at the time of disaster estimated by the estimation unit 104 using the information about the life of the disaster victim. The action for maintaining health of the disaster victims is as described above.

The determination unit 102 determines an action for maintaining health of the disaster victim using the learned model subjected to machine learning. Here, a learned model used for determining an action for maintaining health of the disaster victim is referred to as an action determination model. The action determination model is a model that has learned the relationship between the health condition of the disaster victim and the action for maintaining health of the disaster victim by machine learning. The determination unit 102 uses the health condition of the disaster victim estimated by the estimation unit 104 as an input to the action determination model. Then, the determination unit 102 acquires a determination result of an action for maintaining health of the disaster victim, which is an output of the action determination model. The estimation unit 104 may estimate the health condition of the disaster victim using a predetermined algorithm.

Specific examples of the action determination for maintaining health of the disaster victims will be described. For example, a case where the health condition in which the risk of low back pain is high from the state of joints or muscles is estimated by the estimation unit 104 will be described as an example. At this time, the determination unit 102 can determine an exercise that is effective for a joint or a muscle, which is considered to be a main cause of low back pain, as an action for maintaining health of the disaster victim. In a case where the estimation unit 104 estimates that the mental stress is high, the determination unit 102 may determine stretching of muscles related to respiration as an action for maintaining health of the disaster victim. At this time, the determination unit 102 may determine stretching that is considered to have an effect of alleviating anxiety and stress as an action for maintaining health of the disaster victim.

When the plurality of risks or stresses is estimated to be high by the estimation unit 104, the determination unit 102 can determine an appropriate action for maintaining health of the disaster victim by the action determination model. At this time, the score of each of the plurality of risks or stresses estimated by the estimation unit 104 is input to the action determination model. Then, the action determination model outputs an appropriate action for maintaining health of the disaster victim.

Here, the determination unit 102 may determine a timing and a place at which an action for maintaining health is performed in addition to the content of the action for maintaining health. The determination unit 102 may determine the frequency at which an action for maintaining health is performed. The determination unit 102 can determine the timing, place, and frequency of the action for maintaining health to be appropriate timing, place, and frequency according to the content of the action for maintaining health.

The determination unit 102 may determine one action for maintaining health of the disaster victim. The action for maintaining health of the disaster victim decided by determination unit 102 may be a recommended program including a plurality of actions. In the recommended program, for example, the order in which a plurality of actions is performed is determined. For example, in the recommended program, the timing at which a plurality of actions is performed may be determined. The action for maintaining health of the disaster victim determined by the determination unit 102 may be a candidate for the action performed by the disaster victim. The determination unit 102 can determine candidates for an action for maintaining health of the disaster victim based on the health condition of the disaster victim. At this time, the disaster victim can select an action he/she wants to perform from the candidates.

The determination unit 102 can determine an action for maintaining health of the disaster victim by further using information different from the health condition of the disaster victim, in addition to the health condition of the disaster victim.

For example, the determination unit 102 may determine an action for maintaining health of the disaster victim by using a vehicle interior environment. The vehicle interior environment includes information about the temperature or the humidity in the vehicle 90. Depending on the vehicle interior environment, it may be difficult to perform an action for maintaining health of the disaster victim in the vehicle 90. On the other hand, the vehicle interior environment may be an optimal vehicle interior environment for performing an action for maintenance. Therefore, the determination unit 102 may determine an action for maintaining health of the disaster victim by using the vehicle interior environment.

For example, the determination unit 102 can input information about the vehicle interior environment to the action determination model. Then, the determination unit 102 can acquire an action for maintaining health determined based on the vehicle exterior environment as an output by the action determination model. An appropriate environment may be determined in advance for each action for maintaining health. The determination unit 102 may determine an action in which the vehicle interior environment matches an appropriate environment as an action for maintaining health.

For example, the determination unit 102 may determine an action for maintaining health of the disaster victim by using the vehicle exterior environment. The vehicle exterior environment includes at least one of weather, temperature, and humidity. Depending on the vehicle exterior environment, it may be difficult to perform an action for maintaining health of the disaster victim in the vehicle 90. For example, when the temperature outside the vehicle is too high, it may be difficult for the disaster victim to exercise outside the vehicle. Therefore, the determination unit 102 may determine an action for maintaining health of the disaster victim by using the vehicle exterior environment.

The determination unit 102 may determine an action for maintaining health of the disaster victim using, for example, disaster occurrence information or a disaster damage situation. As described above, the vehicle exterior environment includes at least one of the occurrence information about the disaster or the damage situation due to the disaster. The disaster victim has an action that is difficult to perform depending on the disaster occurrence situation. For example, in the case of a heavy rain disaster, when it continues to rain, it is difficult for the disaster victim to take an action for maintaining health outside the vehicle. Therefore, the determination unit 102 determines an action, for maintaining health, that can be performed in the vehicle 90 by using the occurrence information about the disaster or the damage situation due to the disaster. As the damage caused by the disaster, road conditions and soil conditions around the evacuation place may be bad. At this time, for example, it may be difficult for the disaster victim to walk at a place away from the vehicle 90. Therefore, the determination unit 102 determines an action, for maintaining health, that can be performed in the vehicle 90 or around the vehicle 90 by using the occurrence information about the disaster or the damage situation due to the disaster.

For example, the determination unit 102 can input information about the vehicle exterior environment to the action determination model. Then, the determination unit 102 can acquire an action for maintaining health determined based on the vehicle exterior environment as an output by the action determination model. An appropriate environment may be determined in advance for each action for maintaining health. The determination unit 102 may determine an action in which the vehicle exterior environment matches an appropriate environment as an action for maintaining health. The determination unit 102 may determine an action for maintaining health of the disaster victim by using the vehicle interior environment and the vehicle exterior environment. The determination unit 102 may determine an action for maintaining health of the disaster victim by using information about a status of use of vehicle 90 at the normal time. As described above, the status of use of the vehicle 90 at the normal time includes the driving time or driving frequency at the normal time as described above. The status of use of the vehicle 90 at the normal time includes a break time or a break acquisition interval acquired during driving. A person with a low driving frequency at the normal time may be more likely to feel stress in an evacuation life in the vehicle 90 than a person with a high driving frequency at the normal time. Therefore, the determination unit 102 may determine an action for stress release as an action for maintaining health for a person who drives less frequently at the normal time. The determination unit 102 may determine, as a recommended program that is an action for maintaining health, an action in which the disaster victim goes out of the vehicle and stretches at the same interval as a break acquisition interval at the normal time. The determination unit 102 can determine, as an action for maintaining health, a recommended program for repeatedly performing stretching at a time interval shorter than a predetermined time for a disaster victim whose physical condition deteriorates if driving is continued for the predetermined time at the normal time.

For example, the determination unit 102 can input information about a status of use of the vehicle 90 at the normal time to the action determination model. Then, the determination unit 102 can acquire an action for maintaining health determined based on information about the status of use of the vehicle 90 at the normal time as an output by the action determination model. An appropriate action for maintaining health may be determined in advance with respect to the information about the status of use of the vehicle 90 at the normal time. The determination unit 102 may determine an action related to the information about the status of use of the vehicle 90 at the normal time as an action for maintaining health.

In addition, the determination unit 102 may determine an action for maintaining health of the disaster victim by using information about an article that can be used at the time of disaster. The action for maintaining health includes an action that can obtain a higher effect by using an article. Therefore, the determination unit 102 may determine an action to be performed using an article usable at the time of disaster as an action for maintaining health. As described above, the acquisition unit 101 stores the acquired information about an article that can be used at the time of disaster in the database, for example, in the form of a list. A necessary article and a substitute for the article are determined in advance for each action for maintaining health. That is, information in which an action for maintaining health, a necessary article, and a substitute are associated with each other is stored in, for example, a database. Therefore, the determination unit 102 can determine, as an action for maintaining health, an action in which an article necessary for an action for maintaining health or a substitute article is included in a list of information about an article that can be used at the time of disaster.

A specific example of determining an action for maintaining health of the disaster victim using information about an article that can be used at the time of disaster will be described. An exercise in which a higher effect can be obtained by using the stretch pole will be described as an example. When the disaster victim performs the exercise, there is a low possibility that a stretch pole is placed in the vehicle 90 at the time of disaster. However, a simple substitute for the pole for stretching may be created by winding a bath towel cylindrically. A simple substitute for the pole for stretching can be created by winding a towel around the core of the wrap. For exercise using the stretch pole, it is predetermined that a substitute for the stretch pole is a towel or wrap core. Therefore, in a case where the list of information about an article that can be used at the time of disaster includes a towel or a core of a wrap, exercise that can be effective by using the stretch pole can be determined as an action for maintaining health.

The determination unit 102 may determine an action for maintaining health of the disaster victim using the community information. As described above, the community information includes the frequency of participation in the community. Therefore, the determination unit 102 may determine, as an action for maintaining health, a recommended program for executing exercise at the frequency same as the frequency at which the disaster victim has participated in the community. The community information includes information about participating members of the community to which the disaster victim belongs. For example, the determination unit 102 may determine an exercise with a participating member of a community as an action for maintaining health even at an evacuation place based on position information of the participating member. For example, there is a case where the disaster victims participating in the same community have the same evacuation destination or evacuation destinations are close to each other. Therefore, even at the time of disaster, there is a case where the disaster victim can be refreshed by performing exercise with the participating members of the community in which the disaster victim participates at the normal time.

In addition, the determination unit 102 may determine, as an action for maintaining health, that disaster victims participating in different communities at the normal time exercise together at the time of disaster. When evacuating to a place away from home at the time of a disaster, it may be difficult for a disaster victim to participate in a community to which he/she belongs at the normal time. However, different communities may have the same activity content. Then, for example, there is a case where there is a disaster victim belonging to the communities with the same activity content among the disaster victims evacuating to the same evacuation place. In this case, the determination unit 102 may determine that the disaster victims belonging to the communities with the same activity content gather and exercise as an action for maintaining health.

Here, the action recommendation device 10 may not include the estimation unit 104. For example, the estimation unit 104 may be included in the determination unit 102. The health condition estimation model and the action determination model may be the same model.

The output unit 103 is an aspect of an output means for outputting action information. The output unit 103 outputs information about the action for maintaining health of the disaster victim, which is recommended to the disaster victim. The information about the action for maintaining health of the disaster victims is information necessary for the disaster victim to take an action. The information about the action for maintaining health of the disaster victims is a description of the action. For example, in a case where the action for maintaining health of the disaster victim is exercise, the description of the action may include a description of part of the body to be moved, a direction of movement, and a tip of exercise. The description of the action may include the number of times of performing the action, the time required for the action, the name of the action, and the timing at which the action is performed.

The information about the action for maintaining health of the disaster victims is not limited thereto. When the action for maintaining health of the disaster victim is the recommended program, the information about the action for maintaining health of the disaster victim may include the order in which the action is performed. The information about the action for maintaining health of the disaster victim may include a reason for recommendation of the action. The reason for recommendation is a reason why the determination unit 102 has determined the action. The reason for recommendation may be, for example, the health condition of the disaster victim, which is used by determination unit 102 to decide the action.

The output unit 103 outputs information about the action for maintaining health of the disaster victim in a mode that can be confirmed by the disaster victim. The output unit 103 outputs the information about the action for maintaining health of the disaster victim in a mode that can be confirmed by the disaster victim, whereby the disaster victim can obtain information about the recommended action for maintaining health. The output unit 103 outputs information about the action for maintaining health to a device (not illustrated) installed in the vehicle 90. The device installed in the vehicle 90 may be a device used for the operation of the vehicle 90 at the normal time.

The device installed in the vehicle 90 includes a display device. An example of the display device is a display. A display may be installed in the vehicle 90. The display may be used in a car navigation system at the normal time. The output unit 103 may output, to the display, information about the action for maintaining health of the disaster victim. The display can display information about the action for maintaining health of the disaster victim on the screen. Then, the disaster victim can check the information about the action for maintaining health of the disaster victim by viewing the display of the screen. On the screen of the display, the description of the operation can be displayed by a moving image.

The device installed in the vehicle 90 includes a voice output device. An example of the voice output device is a speaker. The voice output device can output, by voice, information about an action for maintaining health. The voice output device includes a vehicle interior voice output device and a vehicle exterior voice output device. The vehicle interior voice output device may be used as a speaker of a car audio system at the normal time. The vehicle exterior voice output device may be used as a speaker of an alarm device at the normal time.

The output unit 103 may output information about the action for maintaining health to the communication terminal 91. The communication terminal 91 can display information about an action for maintaining health on a screen of the communication terminal 91. The communication terminal 91 can output, by voice, information about an action for maintaining health from a speaker included in the communication terminal 91.

The output destination by the output unit 103 may be different depending on the content of the operation. In a case where the action for maintaining health determined by the determination unit 102 is an action performed outside the vehicle 90, the output unit 103 can output information about the action for maintaining health to the vehicle exterior voice output device. On the other hand, in a case where the action for maintaining health determined by the determination unit 102 is an action performed in the vehicle 90, the output unit 103 can output information about the action for maintaining health to the vehicle interior voice output or the display device.

The output destination by the output unit 103 may be different depending on the location of the disaster victim. When the disaster victim is in the vehicle 90, the output unit 103 can output information about the action for maintaining health to the vehicle interior voice output or the display device. On the other hand, when the disaster victim is outside the vehicle, the output unit 103 can output information about an action for maintaining health to the vehicle exterior voice output device. Whether the disaster victim is in the vehicle can be determined by, for example, the detection result by the sensor 901 installed in the vehicle 90.

The output destination by the output unit 103 is not limited thereto. The number of output destinations by the output unit 103 may be one or plural. For example, the output unit 103 may output information about the action for maintaining health to the display device and the vehicle interior voice output device. Then, the output destination by the output unit 103 can be determined by the determination unit 102, for example.

The timing at which the output unit 103 outputs the information about the action for maintaining health may be set in advance. For example, the output unit 103 may periodically output information about the action for maintaining health. For example, at 12:00 every day, the output unit 103 may output information about the action for maintaining health. The timing at which the output unit 103 outputs information about the action for maintaining health may be set in advance by the disaster victim. The disaster victim can set a timing at which he/she wants to receive information about the action for maintaining health. Then, the output unit 103 may output information about the action for maintaining health at the timing set by the disaster victim.

The timing at which the output unit 103 outputs the information about the action for maintaining health may be determined by the content of the action. As described above, the determination unit 102 determines the timing at which an action for maintaining health is performed. Therefore, the output unit 203 can output information about the action for maintaining health in accordance with the timing at which the action for maintaining health is performed. For example, the output unit 103 may output information about the action for maintaining health a predetermined time before the timing at which the action for maintaining health is performed. The predetermined time is, for example, a time during which a disaster victim can prepare for an action for maintaining health. The predetermined time is a time during which the disaster victim does not forget to take an action for maintaining health. For example, when the timing at which the output unit 103 outputs information is earlier than the timing at which the disaster victim performs an action, the disaster victim may forget to take an action for maintaining health. A specific example of the predetermined time is 30 minutes before, but is not limited thereto.

The timing at which the output unit 103 outputs the information about the action for maintaining health may be determined according to the urgency of the action for maintaining health. For example, when the health condition of the disaster victim is at high risk of economy class syndrome, the determination unit 102 may determine walking outside the vehicle as an action for maintaining health. At this time, when the output of the information by the output unit 103 is delayed, it may be too late. Therefore, in a case where the urgency of the action for maintaining health is high, the output unit 103 may output the information immediately after the determination by the determination unit 102. On the other hand, when the urgency of the action for maintaining health is low, for example, the output unit 103 may output the information at a timing set by the disaster victim.

Furthermore, the timing at which the output unit 103 outputs the information about the action for maintaining health may be determined according to the content of the action for maintaining health and the place where the disaster victim is present. When the action for maintaining health is an action performed outside the vehicle, the output unit 103 may output information about the action for maintaining health at the timing when the disaster victim is outside the vehicle. When the action for maintaining health is an action performed in the vehicle 90, the output unit 103 may output information about the action for maintaining health at the timing when the disaster victim is in the vehicle 90. Whether the disaster victim is in the vehicle 90 can be determined, for example, by the detection result by the sensor 901 installed in the vehicle 90.

The timing at which the output unit 103 outputs the information about the action for maintaining health may be determined by the determination unit 102, for example. The timing at which the output unit 103 outputs the information about the action for maintaining health is not limited thereto. The output by the output unit 103 may be performed once or a plurality of times. In a case where the output unit 103 outputs the information about the action for maintaining health a plurality of times, the output unit 103 may output the information about the action for maintaining health to a different output destination for each output.

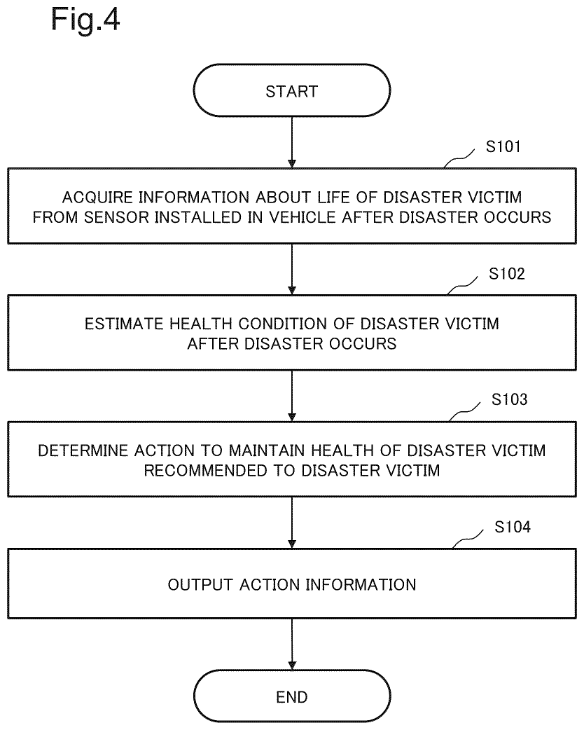

The operation of the action recommendation device 10 including the acquisition unit 101, the determination unit 102, the output unit 103, the acquisition unit 101, and the estimation unit 104 will be described with reference to FIG. 4. FIG. 4 is a flowchart illustrating an operation of the action recommendation device 10.

In step S101, the acquisition unit 101 acquires information about the life of the disaster victim living an evacuation life in the vehicle 90, the information being obtained based on the detection result by the sensor installed in the vehicle 90. In step S102, the estimation unit 104 estimates the health condition of the disaster victim based on information about the life of the disaster victim. In step S103, the determination unit 102 determines an action for maintaining health of the disaster victim, which is recommended to the disaster victim, according to the health condition. In step S104, the output unit 103 outputs information about the action for maintaining health. Then, the action recommendation device 10 terminates the operation.

A case where the action recommendation device 10 includes the acquisition unit 101, the determination unit 102, and the output unit 103 will be described. In this case, after step S101, the determination unit 102 determines an action for maintaining health of the disaster victim to be recommended to the disaster victim, based on information about the life of the disaster victim. Then, the output unit 103 outputs information about an action for maintaining health, and the action recommendation device 10 terminates the operation.

In the present example embodiment, in the action recommendation device 10, the acquisition unit 101 acquires information about the life of the disaster victim living an evacuation life in the vehicle 90, the information being obtained based on the detection result by the sensor installed in the vehicle 90. Then, the determination unit 102 determines an action for maintaining health of the disaster victim, which is recommended to the disaster victim, based on information about the life of the disaster victim. When a disaster occurs, the disaster victim may be evacuated. There is a case where a disaster victim lives an evacuation life in the vehicle 90. At this time, a physical or mental load may be applied to the disaster victim. That is, for example, the disaster victim may be unwell. Therefore, the determination unit 102 determines an action for maintaining health of the disaster victim to be recommended to the disaster victim, based on information about the life of the disaster victim. Then, the output unit 103 outputs information about the action for maintaining health, thereby making it possible to promote health of the disaster victim who is living an evacuation life in the vehicle 90 at the time of disaster. Then, the output unit 103 outputs the information about the action for maintaining health, thereby making it possible to support decision making on the action for maintaining health by the disaster victim.

The acquisition unit 101 acquires information about the life of the disaster victim living an evacuation life in the vehicle 90, the information being obtained based on the detection result by the sensor installed in the vehicle 90. When the disaster victim lives an evacuation life in the vehicle 90, the acquisition unit 101 can acquire information about the life of the disaster victim. That is, there is a case where the disaster victim does not need to perform work such as input of information about living of the disaster victim. Therefore, it is possible to reduce the time and effort of the disaster victim in determining an action for maintaining health of the disaster victim recommended to the disaster victim. After occurrence of a disaster, there are many cases where the disaster victim performs an action such as responding to damage caused by the disaster and procuring food items. In such a situation, it may be difficult for the disaster victim to perform work such as inputting information about the life of the disaster victim. Therefore, it is possible to easily promote the health of the disaster victim by reducing the labor of the disaster victim.

In the action recommendation device 10 according to the present example embodiment, the sensor is a sensor used for operating the vehicle 90 before a disaster occurs. In the vehicle 90, a sensor used for operating the vehicle 90 is installed at the normal time. Examples of the sensor used for the operation of the vehicle 90 are a load sensor, a capacitance sensor, a pressure sensor, or a radar sensor that detects an occupant of the vehicle 90 at the normal time. The sensors used for the operation of the vehicle 90 are not limited thereto. After the occurrence of the disaster, the acquisition unit 101 acquires information about the life of the disaster victim obtained based on the detection results by these sensors. That is, it is not necessary to prepare a new sensor for acquiring information about the life of the disaster victim. Therefore, it is possible to reduce time and effort required for promoting health of the disaster victim.

Since the acquisition unit 101 uses a sensor installed in the vehicle 90 at the normal time, it is possible to acquire information about the life of the disaster victim immediately after the disaster occurs. For example, in a case where a new sensor is prepared to acquire information about the life of the disaster victim, it may be difficult for the acquisition unit 101 to acquire the information about the life of the disaster victim until the new sensor is prepared. However, by using a sensor installed in the vehicle 90, the acquisition unit 101 can acquire information about the life of the disaster victim immediately after the disaster occurs. Therefore, the determination unit 102 can determine an action for maintaining health of the disaster victim based on information about the lives of more disaster victims. As a result, it is possible to promote the health of the disaster victim who is living an evacuation life in the vehicle 90 at the time of disaster.

In the present example embodiment, in the action recommendation device 10, the estimation unit 104 estimates the health condition of the disaster victim based on information about the life of the disaster victim. Then, the determination unit 102 determines an action for maintaining health according to the health condition. There is a case where a health condition of a disaster victim who lives an evacuation life in the vehicle 90 changes. Here, the health condition includes at least one of a risk of pain in the musculoskeletal system, a risk of disorder in the musculoskeletal system, disorder in movement of joints and the like, and mental stress. At the time of disaster, for example, for the disaster victim, the risk of pain or disorder may increase, or the mental stress may increase. Depending on the health condition of the disaster victim, the action that the disaster victim should perform for maintaining health may be different from a desired action. Therefore, the determination unit 102 determines an action for maintaining health according to the health condition of the disaster victim estimated by the estimation unit 104. Therefore, the action recommendation device 10 can recommend an appropriate action according to the health condition of the disaster victim. Then, it is possible to promote the health of the disaster victim at the time of disaster by the disaster victim performing the recommended action.

In the present example embodiment, in the action recommendation device 10, the output unit 203 outputs information about an action for maintaining health to a device installed in the vehicle 90. There is a case where the disaster victim lives an evacuation life in the vehicle 90. Therefore, the output unit 103 outputs the information about the action for maintaining health to the device installed in the vehicle 90, so that the disaster victim can obtain the information about the action for maintaining health during the evacuation life. That is, the disaster victims can save time and effort for obtaining information about the action for maintaining health. The time and effort for obtaining information about the action for maintaining health means, for example, accessing a specific site or visiting a specific place in a shelter in order to obtain information about the action for maintaining health. There is a case where it is easy for a disaster victim to perform an operation for maintaining health by reducing time and effort for obtaining information about the action for maintaining health. As a result, it is possible to promote health of the disaster victim at the time of disaster.

Since the output unit 103 outputs information about an action for maintaining health to a device installed in the vehicle 90, there is a case where a new output device is not necessary. For example, in a case where a new output device is required to output information about an action for maintaining health, it may be difficult for the output unit 103 to output information about an operation for maintaining health until the new output device is prepared. However, the output unit 103 can output the information about the action for maintaining health to the device installed in the vehicle 90, so that the information about the action for maintaining health can be output immediately after the disaster occurs. Therefore, the output unit 103 can output information about the action for maintaining health at an appropriate timing. As a result, it is possible to promote the health of the disaster victim who is living an evacuation life in the vehicle 90 at the time of disaster.

In the action recommendation device 10 according to the present example embodiment, the information about the life of the disaster victim includes at least one of an action history of the disaster victim in the vehicle 90, an action history of the disaster victim outside the vehicle 90, and a physical condition or a mental condition of the disaster victim. There is a case where a disaster victim who lives an evacuation life in the vehicle 90 spends a lot of time in the vehicle 90. After occurrence of a disaster, it may be difficult to leave the evacuation place depending on the damage situation. For this reason, the disaster victim may spend a lot of time around the vehicle. Therefore, the acquisition unit 101 acquires information about the life of the disaster victim including the action history in the vehicle 90 or the action history outside the vehicle 90. Therefore, the estimation unit 104 may be able to accurately estimate the health condition of the disaster victim. Then, the determination unit 102 can determine an appropriate action for maintaining health from the health condition of the disaster victim estimated with high accuracy. As a result, it is possible to promote the health of the disaster victim who is living an evacuation life in the vehicle 90 at the time of disaster.

The action history of the disaster victim in the vehicle 90 includes at least one of the posture in the vehicle, the habit of the posture, the sitting position retention time in the vehicle, the physical activity amount during the sitting position retention, the vital sign, the heart rate fluctuation, and the pulse fluctuation. Since the space where the disaster victim living an evacuation life in the vehicle 90 lives a life is limited to the space inside the vehicle, for example, the posture of a disaster victim in the vehicle may be limited. Since the disaster victim lives an evacuation life in the vehicle 90, there is a case where the time to sit on the seat of the vehicle 90 is long. Therefore, the action history of the disaster victim in the vehicle 90 includes, for example, at least one of a posture in the vehicle, a habit of the posture, and a sitting position retention time in the vehicle. Therefore, the acquisition unit 101 can appropriately acquire how the disaster victim spends in the vehicle. Therefore, the estimation unit 104 may be able to accurately estimate the health condition of the disaster victim. Then, the determination unit 102 can determine an appropriate action for maintaining health from the health condition of the disaster victim estimated with high accuracy. As a result, it is possible to promote the health of the disaster victim who is living an evacuation life in the vehicle 90 at the time of disaster.

In the action recommendation device 10 according to the present example embodiment, the estimation unit 104 estimates the health condition of the disaster victim using the learned model subjected to machine learning. Then, the acquisition unit 101 acquires information about the life of the disaster victim determined according to the importance level estimated by the learned model. For example, the acquisition unit 101 may acquire information with a high importance level and may not acquire information with a low importance level. After the disaster occurs, there is a case where the power that can be consumed is limited. Communication may be restricted due to a disaster. In such an environment, the acquisition unit 101 acquires information about the life of the disaster victim determined according to the importance level of the information. Therefore, it is possible to reduce power consumption and communication volume associated with information acquisition. It is possible to reduce the amount of information used by the estimation unit 104 for estimating the health condition. As a result, the amount of calculation for estimating the health condition can be reduced.

In the action recommendation device 10 according to the present example embodiment, the learned model is a model in which a correlation between information obtained from a detection result by a sensor installed in the vehicle 90 and information obtained from a detection result by a sensor installed in a communication terminal is additionally learned before a disaster occurs. Then, the acquisition unit 101 acquires information about the life of the disaster victim determined according to the correlation. The information having a strong correlation may be information from which similar information can be obtained from either the sensor installed in the vehicle 90 or the sensor 911 installed in the communication terminal 91. Therefore, for example, the acquisition unit 101 can acquire information having a strong correlation from any one of the sensor 901 installed in the vehicle 90 and the sensor 911 installed in the communication terminal 91. The acquisition unit 101 may select a sensor that acquires information in accordance with a remaining battery amount or power consumption for information having a strong correlation. The acquisition unit 101 may switch a sensor that acquires information according to a remaining battery amount or power consumption. As described above, the acquisition unit 101 acquires information having a strong correlation from either the sensor installed in the vehicle 90 or the sensor 911 installed in the communication terminal 91, whereby power consumption and a communication volume associated with information acquisition can be reduced. It is possible to reduce the amount of information used by the estimation unit 104 for estimating the health condition. As a result, the amount of calculation for estimating the health condition can be reduced.

In the present example embodiment, in the action recommendation device 10, when a plurality of disaster victims uses the same vehicle 90, the acquisition unit 101 distinguishes information about the life of the disaster victims using the body weights of the disaster victims detected by the sensor. At the time of disaster, a plurality of disaster victims may evacuate to one vehicle 90. At this time, the acquisition unit 101 is required to distinguish which disaster victim's information the acquired information about the life of the disaster victim is. Therefore, the acquisition unit 101 can distinguish which disaster victim's information the acquired information about the life of the disaster victim is by using the weight of the disaster victim detected by the sensor. As a result, the estimation unit 104 may be able to accurately estimate the health condition of each disaster victim. Then, the determination unit 102 can determine an appropriate action for maintaining health for each of the disaster victims from the health condition of each of the disaster victims estimated with high accuracy. As a result, it is possible to promote health of a plurality of disaster victims who lives an evacuation life in the vehicle 90 at the time of disaster.

In the present example embodiment, in the action recommendation device 10, the acquisition unit 101 acquires environmental information outside the vehicle including occurrence information of a disaster or a damage situation due to the disaster. The determination unit 102 determines an action for maintaining health according to the occurrence information or the damage situation. The disaster victim has an action that is difficult to perform depending on the disaster occurrence situation. For example, in the case of a heavy rain disaster, it may continue to rain. As the damage caused by the disaster, road conditions and soil conditions around the evacuation place may be bad. In such a case, it is difficult for the disaster victim to take an action for maintaining health outside the vehicle. Therefore, the determination unit 102 determines an action, for maintaining health, that can be performed in the vehicle using the occurrence information about the disaster or the damage situation due to the disaster. Therefore, the disaster victim can perform an action for maintaining health. As a result, it is possible to promote the health of the disaster victim who is living an evacuation life in the vehicle 90 at the time of disaster.

In the present example embodiment, in the action recommendation device 10, the acquisition unit 101 acquires information about an article that can be used by a disaster victim after occurrence of a disaster. Then, the determination unit 102 further determines an action for maintaining health by using information about usable articles. The action for maintaining health includes an action that can obtain a higher effect by using an article. Therefore, the determination unit 102 determines an action to be performed using an article usable at the time of disaster as an action for maintaining health. A disaster victim who has performed an action for maintaining health using the article may obtain a high effect as compared with a case where an action for maintaining health is performed without using an article. As a result, it is possible to promote the health of the disaster victim who is living an evacuation life in the vehicle 90 at the time of disaster.

The acquisition unit 101 acquires information about an article that can be used by the disaster victim after the disaster. Then, the determination unit 102 can determine an exercise using the article as an action for maintaining health. At this time, there is a case where the disaster victim does not need to newly search for or purchase an article in order to perform an action for maintaining health. That is, it is possible to reduce the time and effort of the disaster victim to perform the recommended action for maintaining health. There is a case where it is easy for a disaster victim to take an action for maintaining health. As a result, it is possible to promote the health of the disaster victim who is living an evacuation life in the vehicle 90 at the time of disaster.

### Second Example Embodiment

The second example embodiment will be described in detail with reference to the drawings. Hereinafter, description of content overlapping with the above description will be omitted to the extent that the description of the present example embodiment is not unclear.

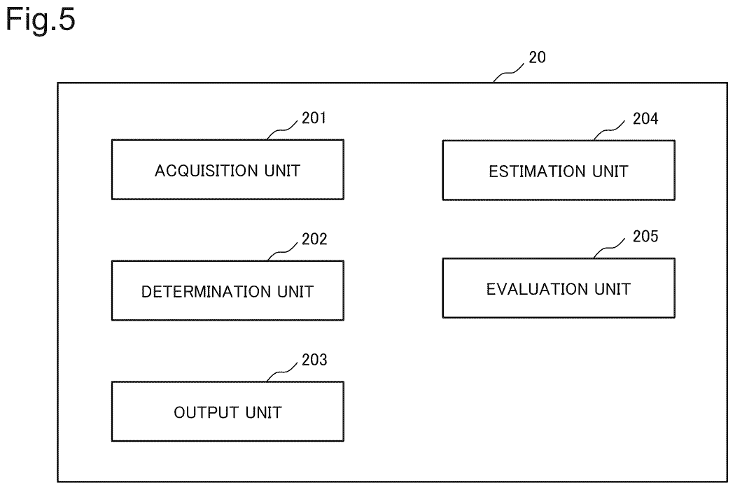

A configuration of an action recommendation device 20 will be described with reference to FIG. 5. FIG. 5 is a block diagram illustrating an example of a configuration of the action recommendation device 20. The action recommendation device 20 includes an acquisition unit 201, a determination unit 202, an output unit 203, and an evaluation unit 205. The action recommendation device 20 may include an estimation unit 204.

The acquisition unit 201, the determination unit 202, the output unit 203, and the estimation unit 204 are similar to those in the first example embodiment. That is, the acquisition unit 101 is related to the acquisition unit 201, the determination unit 102 is related to the determination unit 202, the output unit 103 is related to the output unit 203, and the estimation unit 104 is related to the estimation unit 204. The action recommendation device 20 further includes the evaluation unit 205 in addition to the configuration included in the action recommendation device 10.

The evaluation unit 205 is an aspect of an evaluation means for evaluating an action for maintaining health performed by a disaster victim. There is a case where the disaster victim performs an action, for maintaining health, the information about which the output unit 203 outputs. At this time, the evaluation unit 205 can evaluate the action for maintaining health performed by the disaster victim. Then, the output unit 203 can output content related to the evaluation of the action for maintaining health of the disaster victim. That is, the action recommendation device 20 can perform feedback or the like on the action performed by the disaster victim.

First, the acquisition unit 201 acquires a state in which the disaster victim performs an action for maintaining health. Acquisition unit 201 acquires a state captured by a camera installed in the vehicle 90, in which the disaster victim performs an action for maintaining health. When the disaster victim performs an action for maintaining health outside the vehicle 90, the state of the disaster victim is imaged by the vehicle exterior imaging camera. When the disaster victim performs an action for maintaining health in the vehicle 90, the state of the disaster victim is imaged by the vehicle interior imaging camera. The acquisition unit 201 may acquire, from the sensor 911 installed in the communication terminal 91, a state in which the disaster victim performs an action for maintaining health.

Then, the evaluation unit 205 evaluates the action for maintaining health performed by the disaster victim, which is acquired by the acquisition unit 201. The evaluation unit 205 may evaluate whether the disaster victims are performing the action for maintaining health. For example, in a case where the action for maintaining health is a program, the evaluation as to whether the disaster victim is performing the action for maintaining health may be expressed by a ratio performed by the disaster victim.

The evaluation unit 205 may evaluate whether the disaster victims are appropriately performing the action for maintaining health. The evaluation unit 205 may express the appropriateness of the action performed by the disaster victim by, for example, a rank. For example, in a case where the appropriate action has been performed, the rank may be represented by rank 5, and in a case where the appropriate action has not been performed, the rank may be represented by rank 1. The evaluation unit 205 may express the appropriateness of the action performed by the disaster victim by a ratio. The evaluation unit 205 may evaluate the action performed by the disaster victim for each body part. For example, in a case where the action for maintaining health is an exercise of moving a hand and a foot, the evaluation unit 205 may evaluate each of the movement of the hand and the movement of the foot.

Known techniques can be used to evaluate whether the disaster victim is performing the action for maintaining health and whether the disaster victim is appropriately performing the action for maintaining health. The evaluation unit 205 may evaluate the action for maintaining health performed by the disaster victim by acquiring the evaluation on itself by the disaster victim. The evaluation unit 205 may evaluate an action for maintaining health performed by the disaster victim by acquiring an evaluation on the disaster victim by a third party.

The evaluation unit 205 may evaluate whether there is an effect by the disaster victim performing an action for maintaining health. For example, the estimation unit 104 can estimate the health condition of the disaster victim based on the state of the disaster victim performing the action for maintaining health, which is acquired by the acquisition unit 101. The method of estimating the health condition is as described above. Then, the evaluation unit 205 compares the health conditions before and after the disaster victim performs an action for maintaining health. The evaluation unit 205 can evaluate the effect of an action for maintaining health from the comparison result. The method of evaluating the effect of the disaster victims performing the action for maintaining health is not limited to this. Known techniques can be used to evaluate the effect of the disaster victims performing the action for maintaining health.

The evaluation unit 205 may express the effect of the action performed by the disaster victim by, for example, a rank. For example, in a case where the effect is large, the rank may be represented by rank 5, and in a case where the effect is small, the rank may be represented by rank 1. The evaluation unit 205 may express the effect of the action performed by the disaster victim by a ratio. The evaluation unit 205 may evaluate the effect of the action performed by the disaster victim for each body part. For example, in a case where the action for maintaining health is an exercise of moving a hand and a foot, the evaluation unit 205 may evaluate each of the effect on the hand and the effect on the foot.

The evaluation content by the evaluation unit 205 is not limited thereto. The evaluation unit 205 can perform evaluation according to the disaster victim and the action for maintaining health.

The output unit 203 outputs the content related to the evaluation of the action for maintaining health of the disaster victim. The output unit 203 may output an improvement point for the action for maintaining health performed by the disaster victim. A known technique can be used to determine the content of the improvement point to be output to the disaster victim.

The output unit 203 may output an action for maintaining health different from the action for maintaining health already output by the output unit 103 to the disaster victim. There is a case where it is evaluated that there is the enough effect obtained by the implementation of the action for maintaining health by the disaster victim suffices. At this time, there is a case where it is possible to further promote health by the disaster victim performing an action different from the action. On the other hand, when it is evaluated that the disaster victim cannot appropriately perform an action for maintaining health, the action may be difficult for the disaster victim. At this time, there is a case in which the output unit 203 should output an action for maintaining health with a reduced difficulty level.

Therefore, the output unit 203 may output a different action for maintaining health to the disaster victim. At this time, the different action for maintaining health output by the output unit 203 may be determined by the determination unit 202.

The output unit 203 may output a notification including the situation of the disaster victim to a preset notification destination. The situation of the disaster victim includes at least one of the health condition of the disaster victim, the action for maintaining health of the disaster victim, and the evaluation content by the evaluation unit 205. The situation of the disaster victim may include information about the evacuation place for the disaster victim. The notification output by the output unit 203 may include a visit request of the disaster victim, a medical care request and a treatment request of the disaster victim, and the like. The output unit 203 may output the notification to a preset notification destination according to the health condition estimated by the estimation unit 204. Depending on the state or health condition of the disaster victim, for example, it may be necessary for a government or a medical institution to take measures. Therefore, the output unit 203 may output the notification to a preset notification destination based on the state of the disaster victim acquired by the acquisition unit 101 or the health condition estimated by the estimation unit 204. The notification destination set in advance is, for example, a department in charge of a municipality, a medical institution, a primary doctor or nurse, or the like. The condition of the disaster victim includes the health condition of the disaster victim.

Here, the content according to the evaluation output by the output unit 203 may be determined by the determination unit 202. For example, output content by the output unit 203 is determined in advance according to the evaluation by the evaluation unit 205. Therefore, the determination unit 202 can determine the content according to the evaluation output by the output unit 203 based on the evaluation by the evaluation unit 205.

The evaluation unit 205 may evaluate the action of the disaster victim for maintaining health at the normal time. At this time, the acquisition unit 201 acquires a state of the action for maintaining health of the disaster victim at the normal time. The evaluation content and the evaluation method by the evaluation unit 205 are the same as those at the time of disaster. The evaluation unit 205 may store the evaluation at the normal time in, for example, a database.

At the time of disaster, the determination unit 202 may determine an action for maintaining health by using the evaluation at the normal time. For example, a case where the determination unit 202 determines an exercise performed in a community as an action for maintaining health will be described as an example. At this time, for example, the determination unit 202 may determine an exercise in a community including disaster victim, the exercise being highly evaluated at the normal time, as an action for maintaining health.

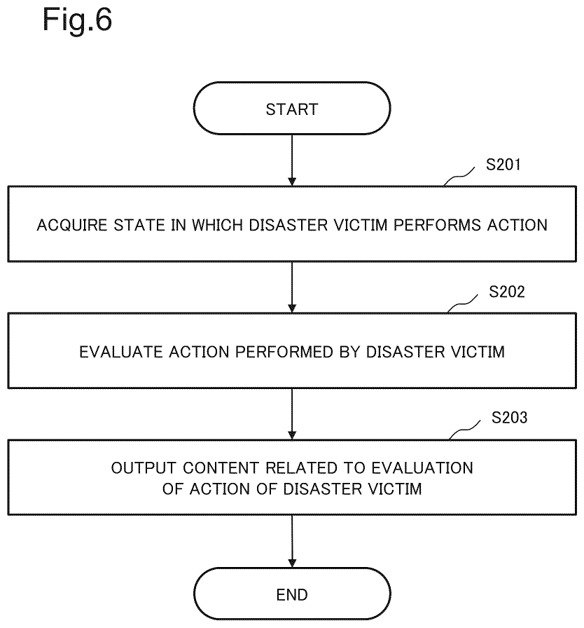

The operation of the action recommendation device 10 including the acquisition unit 201, the determination unit 202, the output unit 203, the estimation unit 104, and the evaluation unit 205 will be described with reference to FIG. 6. FIG. 6 is a flowchart illustrating an operation of the action recommendation device 20. FIG. 6 is a flowchart illustrating an operation of the action recommendation device 20 in a case where a disaster victim performs an action for maintaining health, the action being output by the output unit 203. That is, FIG. 6 is a flowchart illustrating the operation after the action recommendation device 10 performs the operation illustrated in FIG. 4.

In step S104 in FIG. 4, the output unit 103 outputs information about the action for maintaining health. The disaster victim may perform an action for maintaining health. Therefore, in step S201, the acquisition unit 101 acquires a state in which the disaster victim performs an action for maintaining health. In step S202, the evaluation unit 205 evaluates the action for maintaining health performed by the disaster victim. Then, the output unit 203 outputs the content related to the evaluation of the action for maintaining health of the disaster victim. Then, the action recommendation device 10 terminates the operation. Here, the content output by the output unit 203 may be determined by the determination unit 202. That is, after step S202, the determination unit 202 determines the content to be output according to the evaluation of the action for maintaining health of the disaster victim. Then, in step S203, the output unit 203 may output the content related to the evaluation of the action for maintaining health of the disaster victim.

In the present example embodiment, in the action recommendation device 10, the evaluation unit 205 evaluates an action for maintaining health performed by the disaster victim. Then, the output unit 203 outputs the content related to the evaluation of the action for maintaining health performed by the disaster victim. There is a case where the disaster victim performs an action, for maintaining health, the information about which the output unit 203 outputs. Therefore, the evaluation unit 205 evaluates an action for maintaining health by the disaster victim, and the output unit 203 outputs the content related to the evaluation. Therefore, it is possible to provide feedback on the action for maintaining health of the disaster victim.

An example of the content output according to the evaluation is to output an improvement point of the action for maintaining health for the disaster victim. When the disaster victim checks the improvement point, the disaster victim may be able to perform an appropriate action for maintaining health. As a result, it is possible to effectively promote the health of the disaster victim living an evacuation life in the vehicle 90 at the time of disaster.

Another example of the content output according to the evaluation is to output information about the different action for maintaining health. The evaluation unit 205 may evaluate that the effect of the action for maintaining health by the disaster victim suffices. At this time, there is a case where it is possible to further promote health by the disaster victim performing an action different from the action. On the other hand, when it is evaluated that the disaster victim cannot appropriately perform an action for maintaining health, the action may be difficult for the disaster victim. At this time, there is a case in which the output unit 203 should output an action for maintaining health with a reduced difficulty level. That is, the output unit 103 outputs the different action for maintaining health according to the evaluation, whereby the health of the disaster victim can be efficiently promoted.

Further, another example of the content output according to the evaluation is to output a notification to a preset notification destination. Depending on the state or health condition of the disaster victim, for example, it may be necessary for a government or a medical institution to take measures. Therefore, there is a case where the output unit 203 outputs a notification to a preset notification destination, so that the disaster victim can receive an appropriate response. For example, a government or a medical institution as a notification destination can grasp the situation of the disaster victim. As a result, for example, a government or a medical institution can promptly visit or treat the disaster victim.

In the present example embodiment, in the action recommendation device 10, the acquisition unit 201 acquires a state in which a disaster victim imaged by a camera installed in the vehicle 90 performs an action for maintaining health after occurrence of a disaster. Then, the evaluation unit 205 evaluates the acquired action. There is a case where a disaster victim who lives an evacuation life in the vehicle 90 performs an action for maintaining health in the vehicle 90 or in the vicinity of the vehicle. Therefore, the acquisition unit 201 can acquire a state in which the disaster victim performs an action for maintaining health by a camera installed in the vehicle 90. Therefore, it is possible to reduce time and effort for imaging a situation in which a disaster victim performs an action for maintaining health. The camera is installed in the vehicle 90 from the normal time. For this reason, the disaster victim does not need to prepare a new device in order to image the situation in which the disaster victim performs the action for maintaining health. Therefore, it is possible to reduce time and effort required for promoting health of the disaster victim.

### Hardware Configuration Example

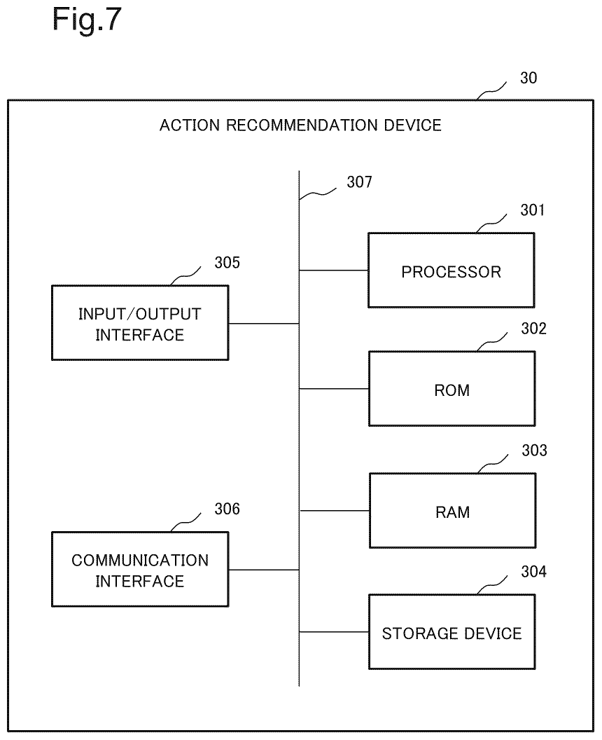

FIG. 7 is a diagram illustrating a hardware configuration example of an action recommendation device 30 according to the present disclosure. The action recommendation device 30 is achieved by a computer. The action recommendation device 30 is an example in a case where the action recommendation device 10 or the action recommendation device 20 is implemented by a computer.

The action recommendation device 30 includes a processor 301, a read only memory (ROM) 302, a random access memory (RAM) 303, a storage device 304 such as a hard disk that stores a program, an input/output interface 305 that inputs and outputs data, and a communication interface 306 for network connection. The components are connected via a bus 307.

The processor 301 operates an operating system to control the entire computer. Examples of the processor 301 include a central processing unit (CPU), a digital signal processor (DSP), and a graphics processing unit (GPU). The processor 301 loads a program stored in, for example, the ROM 302, the storage device 304, or the like. Then, the processor 301 executes each process coded in the program. The processor 301 may execute processes or instructions in the flowchart illustrated based on the program.

The ROM 302 stores an application program, a program according to each embodiment, and the like. The RAM 303 is used as a work area of the processor 301.

Examples of the storage device 304 include a semiconductor memory such as a flash memory, a hard disk drive (HDD), and the like. The storage device 304 stores, for example, an operating system (OS) program, an application program, a program according to each example embodiment, and the like.

The input/output interface 305 is connected to a peripheral device (not illustrated). The connection method may be a wired network or a wireless network.

The communication interface 306 is connected to a communication network (not illustrated) such as a local network (LAN) or a wide area network (WAN) through a wireless or wired network. The communication network may include a plurality of communication networks. As a result, the computer is connected to an external device via the communication network. The action recommendation device 30 may include components other than those illustrated in FIG. 7. For example, the action recommendation device 30 may include a drive device or the like. For example, the processor 301 may be mounted on a drive device or the like, and may read a program or data stored in a non-transitory tangible recording medium into the RAM 303.

While the present disclosure has been particularly shown and described with reference to each of example embodiments, the present disclosure is not limited to the above example embodiments. Various modifications that can be understood by those of ordinary skill in the art can be made to the configuration and details of the present disclosure within the scope of the present disclosure. The configurations in the respective example embodiments can be combined with each other without departing from the scope of the present disclosure.

Further, it is noted that the inventor's intent is to retain all equivalents of the claimed disclosure even if the claims are amended during prosecution.

Some or all of the above example embodiments may be described as the following Supplementary Notes, but are not limited to the following.

### (Supplementary Note 1)

An action recommendation device including
an acquisition means for acquiring information about a life of a disaster victim living an evacuation life in a vehicle, the information being obtained based on a detection result by a sensor installed in the vehicle,
a determination means for determining an action for maintaining health of the disaster victim, the action being recommended to the disaster victim, based on information about the life of the disaster victim, and
an output means for outputting information about the action for maintaining health.

### (Supplementary Note 2)

The action recommendation device according to Supplementary Note 1, wherein
the sensor is a sensor used for operating the vehicle before a disaster occurs

### (Supplementary Note 3)

The action recommendation device according to Supplementary Note 2, wherein
the sensor is a load sensor that detects an occupant of the vehicle before the disaster occurs.

### (Supplementary Note 4)

The action recommendation device according to Supplementary Note 2 or 3, wherein
the sensor is a capacitance sensor that detects an occupant of the vehicle before the disaster occurs.

### (Supplementary Note 5)

The action recommendation device according to any one of Supplementary Notes 2 to 4, wherein the sensor is a pressure sensor that detects an occupant of the vehicle before the disaster occurs.

### (Supplementary Note 6)

The action recommendation device according to any one of Supplementary Notes 2 to 5, wherein
the sensor is a radar sensor that detects an occupant of the vehicle before the disaster occurs.

### (Supplementary Note 7)

The action recommendation device according to any one of Supplementary Notes 1 to 6, further including
an estimation means for estimating a health condition of the disaster victim based on information about the life of the disaster victim, wherein the determination means determines the action according to the health condition.

### (Supplementary Note 8)

The action recommendation device according to Supplementary Note 7, wherein
the health condition represents a physical condition of the disaster victim living the evacuation life in the vehicle.

### (Supplementary Note 9)

The action recommendation device according to Supplementary Note 7 or 8, wherein
the health condition includes at least one of a risk of pain in the musculoskeletal system, a risk of disorder in the musculoskeletal system, disorder in movement of joints and the like, and mental stress.

### (Supplementary Note 10)

The action recommendation device according to any one of Supplementary Notes 7 to 9, wherein
the acquisition means acquires information about a life of the disaster victim before a disaster occurs, and
the estimation means further estimates a health condition of the disaster victim based on information about a life of the disaster victim before the disaster occurs

### (Supplementary Note 11)

The action recommendation device according to Supplementary Note 10, wherein
the acquisition means acquires a status of use of the vehicle by the disaster victim before the disaster occurs, and
the determination means determines an action for maintaining health of the disaster victim according to the status of use.

### (Supplementary Note 12)

The action recommendation device according to any one of Supplementary Notes 1 to 11, wherein
the output means outputs information about the action for maintaining health to a device installed in the vehicle.

### (Supplementary Note 13)

The action recommendation device according to any one of Supplementary Notes 1 to 12, wherein
the output means outputs information about an action for maintaining health at a timing related to urgency of an action for maintaining health.

### (Supplementary Note 14)

The action recommendation device according to any one of Supplementary Notes 1 to 13, wherein
information about a life of the disaster victim includes at least one of an action history of the disaster victim in the vehicle, an action history of the disaster victim outside the vehicle, and a physical condition or a mental condition of the disaster victim.

### (Supplementary Note 15)

The action recommendation device according to Supplementary Note 14, wherein
the action history of the disaster victim in the vehicle includes at least one of a posture in the vehicle, a habit of the posture, a sitting position retention time in the vehicle, a physical activity amount during a sitting position retention, a vital sign, a heart rate fluctuation, and a pulse fluctuation.

### (Supplementary Note 16)

The action recommendation device according to Supplementary Note 14 or 15, wherein
the acquisition means acquires the physical condition based on an image captured by a camera for capturing an outside of the vehicle.

### (Supplementary Note 17)

The action recommendation device according to any one of Supplementary Notes 14 to 16, wherein
the acquisition means acquires the mental condition based on the action history of the disaster victim in the vehicle.

### (Supplementary Note 18)

The action recommendation device according to any one of Supplementary Notes 1 to 17, further including
an evaluation means for evaluating the action for maintaining health performed by the disaster victim, wherein
the output means outputs content related to the evaluation.

### (Supplementary Note 19)

The action recommendation device according to Supplementary Note 18, wherein
the acquisition means acquires a state of the disaster victim performing an action for maintaining health, the state being captured by a camera installed in the vehicle after occurrence of the disaster, and
the evaluation means evaluates the acquired action.

### (Supplementary Note 20)

The action recommendation device according to Supplementary Note 18 or 19, wherein
the output means outputs an improvement point according to the evaluation.

### (Supplementary Note 21)

The action recommendation device according to any one of Supplementary Notes 18 to 20, wherein
the determination means determines a different action for maintaining health according to the evaluation, and
the output means outputs information about the different action for maintaining health.

### (Supplementary Note 22)

The action recommendation device according to any one of Supplementary Notes 18 to 21, wherein
the output means outputs a notification to a notification destination set in advance according to the evaluation.

### (Supplementary Note 23)

The action recommendation device according to Supplementary Note 7, wherein
the estimation means estimates a health condition of a disaster victim using a learned model subjected to machine learning, and
the acquisition means acquires information about a life of the disaster victim, the information being determined according to an importance level estimated by the learned model.

### (Supplementary Note 24)

The action recommendation device according to Supplementary Note 23, wherein
the learned model is a model in which a correlation between information obtained from a detection result by a sensor installed in a vehicle and information obtained from a detection result by a sensor installed in a communication terminal is additionally learned before a disaster occurs, and
the acquisition means acquires information about a life of a disaster victim, the information being determined according to the correlation.

### (Supplementary Note 25)

The action recommendation device according to any one of Supplementary Notes 1 to 24, wherein
when a plurality of the disaster victims uses the same vehicle,
the acquisition means distinguishes information about a life of the disaster victim using a weight of the disaster victim detected by the sensor.

### (Supplementary Note 26)

The action recommendation device according to any one of Supplementary Notes 1 to 25, wherein
the acquisition means acquires environmental information outside the vehicle.

### (Supplementary Note 27)

The action recommendation device according to Supplementary Note 26, wherein
the environmental information outside the vehicle includes occurrence information of a disaster or a damage situation due to the disaster after the disaster occurs.

### (Supplementary Note 28)

The action recommendation device according to Supplementary Note 27, wherein
the determination means determines an action for maintaining health according to the occurrence information or the damage situation.

### (Supplementary Note 29)

The action recommendation device according to any one of Supplementary Notes 1 to 28, wherein
the acquisition means acquires information about an article usable by the disaster victim after a disaster occurs, and
the determination means further determines an action for maintaining health by using information of the usable article.

### (Supplementary Note 30)

An action recommendation method including
acquiring information about a life of a disaster victim living an evacuation life in a vehicle, the information being obtained based on a detection result by a sensor installed in the vehicle,
determining an action for maintaining health of the disaster victim, the action being recommended to the disaster victim, based on information about the life of the disaster victim, and
outputting information about the action for maintaining health.

### (Supplementary Note 31)

A program for causing a computer to execute the steps of
acquiring information about a life of a disaster victim living an evacuation life in a vehicle, the information being obtained based on a detection result by a sensor installed in the vehicle,
determining an action for maintaining health of the disaster victim, the action being recommended to the disaster victim, based on information about the life of the disaster victim, and
outputting information about the action for maintaining health.

### (Supplementary Note 32)

A recording medium storing a program for causing a computer to execute the steps of
acquiring information about a life of a disaster victim living an evacuation life in a vehicle, the information being obtained based on a detection result by a sensor installed in the vehicle,
determining an action for maintaining health of the disaster victim, the action being recommended to the disaster victim, based on information about the life of the disaster victim, and
outputting information about the action for maintaining health.

Some or all of the configurations described in Supplementary Notes 2 to 29 dependent on the above-described Supplementary Note 1 can also be dependent on Supplementary Notes 30 to 32 by the dependency relationship similar to that of Supplementary Notes 2 to 29. Furthermore, some or all of the configurations described as the Supplementary Notes can be similarly dependent on not only the Supplementary Notes 1, and 30 to 32, but also various pieces of hardware and software, and various recording devices or systems for recording software without departing from the above-described example embodiments.

### REFERENCE SIGNS LIST

10, 20, 30 action recommendation device
101, 201 acquisition unit
102, 202 determination unit
103, 203 output unit
104, 204 estimation unit
205 evaluation unit
301 processor
302 ROM
303 RAM
304 storage device
305 input/output interface
306 communication interface
307 bus
90 vehicle
901 sensor
91 communication terminal
911 sensor

1. An action recommendation device comprising:
at least one memory configured to store instructions; and
at least one processor configured to execute the instructions to:
acquire information about a life of a disaster victim living an evacuation life in a vehicle, the information being obtained based on a detection result by a sensor installed in the vehicle;
determine an action for maintaining health of the disaster victim, the action being recommended to the disaster victim, based on information about the life of the disaster victim; and
output information about the action for maintaining health.

2. The action recommendation device according to claim 1, wherein
the sensor is a sensor used for operating the vehicle at a normal time.

3. The action recommendation device according to claim 2, wherein
the sensor is a load sensor that detects an occupant of the vehicle before a disaster occurs.

4. The action recommendation device according to claim 1, wherein the at least one processor is further configured to execute the instructions to:
estimate a health condition of the disaster victim based on information about the life of the disaster victim; and
determine the action for maintaining health according to the health condition.

5. The action recommendation device according to claim 4, wherein
the health condition represents a physical condition of the disaster victim living the evacuation life in the vehicle.

6. The action recommendation device according to claim 4, wherein the at least one processor is further configured to execute the instructions to:
acquire information about the life of the disaster victim at a normal time; and
estimate the health condition of the disaster victim based on the information about the life of the disaster victim at the normal time.

7. The action recommendation device according to claim 6, wherein the at least one processor is further configured to execute the instructions to:
acquire a status of use of the vehicle by the disaster victim before the disaster occurs; and
determine the action for maintaining health of the disaster victim according to the status of use.

8. The action recommendation device according to claim 1, wherein the at least one processor is further configured to execute the instructions to:
output information about the action for maintaining health to a device installed in the vehicle.

9. The action recommendation device according to claim 1, wherein the at least one processor is further configured to execute the instructions to:
output information about the action for maintaining health at a timing related to urgency of the action for maintaining health.

10. The action recommendation device according to claim 1, wherein
information about the life of the disaster victim includes at least one of an action history of the disaster victim in the vehicle, an action history of the disaster victim outside the vehicle, and a physical condition or a mental condition of the disaster victim.

11. The action recommendation device according to claim 10, wherein the at least one processor is further configured to execute the instructions to:
acquire the physical condition based on an image captured by a camera for capturing an outside of the vehicle.

12. The action recommendation device according to claim 1, wherein the at least one processor is further configured to execute the instructions to:
evaluate the action for maintaining health performed by the disaster victim; and
output content related to the evaluation.

13. The action recommendation device according to claim 12, wherein the at least one processor is further configured to execute the instructions to:
output an improvement point according to the evaluation.

14. The action recommendation device according to claim 12, wherein the at least one processor is further configured to execute the instructions to:
determine a different action for maintaining health according to the evaluation; and output information about the different action for maintaining health.

15. The action recommendation device according to claim 4, wherein the at least one processor is further configured to execute the instructions to:
estimate the health condition of the disaster victim using a learned model subjected to machine learning; and
acquire information about the life of the disaster victim, the information being determined according to an importance level estimated by the learned model.

16. The action recommendation device according to claim 15, wherein
the learned model is a model in which a correlation between information obtained from a detection result by a sensor installed in a vehicle and information obtained from a detection result by a sensor installed in a communication terminal is additionally learned before a disaster occurs, and
the at least one processor is further configured to execute the instructions to:
acquire information about the life of the disaster victim determined according to the correlation.

17. The action recommendation device according to claim 1, wherein the at least one processor is further configured to execute the instructions to:
acquire environmental information outside the vehicle including occurrence information of a disaster or a damage situation due to the disaster after the disaster occurs.

18. The action recommendation device according to claim 1, wherein the at least one processor is further configured to execute the instructions to:
acquire information about an article usable by the disaster victim after a disaster occurs; and
determine the action for maintaining health using the information of the usable article.

19. An action recommendation method comprising:
acquiring information about a life of a disaster victim living an evacuation life in a vehicle, the information being obtained based on a detection result by a sensor installed in the vehicle;
determining an action for maintaining health of the disaster victim, the action being recommended to the disaster victim, based on information about the life of the disaster victim; and
outputting information about the action for maintaining health.

20. A non-transitory computer-readable recording medium that records a program for causing a computer to execute:
acquiring information about a life of a disaster victim living an evacuation life in a vehicle, the information being obtained based on a detection result by a sensor installed in the vehicle;
determining an action for maintaining health of the disaster victim, the action being recommended to the disaster victim, based on information about the life of the disaster victim; and
outputting information about the action for maintaining health.
